(19) United States
(12) Patent Application Publication
(10) Pub. No.: US 2024/0327370 A1
(43) Pub. Date: Oct. 3, 2024

(54) PROCESS FOR THE BIOBASED SYNTHESIS OF SCHWEINFURTHINS G, K AND R

(71) Applicants: CENTRE NATIONAL DE LA RECHERCHE SCIENTIFIQUE, Paris (FR); VAST-VIETNAM ACADEMY OF SCIENCE AND TECHNOLOGY, Hanoi (VN)

(72) Inventors: Fanny ROUSSI, Gif-sur-Yvette (FR); Marc LITAUDON, Gif-sur-Yvette (FR); Sandy DESRAT, Gif-sur-Yvette (FR); Gwenaëlle JEZEQUEL, Gif-sur-Yvette (FR); Céline RAMPAL, Gif-sur-Yvette (FR); Van Cuong PHAM, Hanoi (VN); Thi Mai Huong DOAN, Hanoi (VN)

(21) Appl. No.: 18/575,408

(22) PCT Filed: Jun. 30, 2022
(86) PCT No.: PCT/EP2022/068193

§ 371 Date: Dec. 29, 2023

(30) Foreign Application Priority Data
Jul. 1, 2021 (FR) FR2107159

(51) Int. Cl. C07D 311/80; C07D 311/58

(52) U.S. Cl. CPC C07D 311/80; C07D 311/58

(57) ABSTRACT

The present invention relates to a process for preparing at least one schweinfurthin chosen from schweinfurthin G of formula (SW-G), schweinfurthin K of formula (SW-K) and schweinfurthin R of formula (SW-R), characterized in that it comprises a step of using mappain of formula (IV).

### TECHNICAL FIELD

The present invention relates to a novel process for schweinfurthins G, K and R from the biobased precursor constituted by mappain.

The present invention also relates to schweinfurthin R, a novel compound within the schweinfurthin family.

### PRIOR ART

Schweinfurthins are natural products, originally isolated from plants of the genus Macaranga (Euphorbiaceae). They have powerful and selective cytotoxic activity on the National Cancer Institute panel of 60 human cancer cell lines and are particularly active on glioblastoma, kidney and certain leukaemia lines (acute lymphoblastic leukaemia or myeloma). Their cytotoxicity profile does not bear any resemblance to the profiles of molecules currently used in anticancer chemotherapy and thus indicates that they act on one or more new biological targets, which makes them extremely attractive molecules.

As an alternative to extraction, synthetic routes using commercial products have been developed to obtain schweinfurthins. However, said routes involve a significant number of synthetic steps.

Several routes for the total synthesis of schweinfurthin derivatives are described, for example, in patent application WO 2005/092878. However, said synthesis comprises from 16 to 22 synthetic steps with an overall yield of less than 1%. In addition, a route for the total synthesis of schweinfurthin G is described in J. Org. Chem. 2008, 73, 7963-7970, but this route comprises a total of 15 steps for an overall yield of 1%.

Thus, the study and development of the schweinfurthins in general, and more particularly of schweinfurthins G, K and R, are currently curbed by the difficulty of accessing the bio-resource, the low yield of extraction thereof, and the low yield and prohibitive number of steps involved in the chemical synthesis alternatives. In other words, chemical exploration capable of meeting the pharmacological challenges is currently difficult due to the poor accessibility of the schweinfurthins.

### DESCRIPTION OF THE INVENTION

Thus, the need remains for a process for the synthesis of schweinfurthins G, K and R in a reduced number of synthetic steps so as to improve the overall yield thereof.

There is also a need to be able to synthesize many novel schweinfurthin analogues in order to study the properties for the purpose of developing novel therapeutic agents.

There is also a need for a biobased precursor for the synthesis of schweinfurthins G, K and R, and more particularly of schweinfurthin G.

The invention is specifically directed towards meeting these needs.

### SUMMARY OF THE INVENTION

According to a first of its aspects, the present invention relates to a process for preparing at least one schweinfurthin chosen from schweinfurthin G of formula (SW-G),

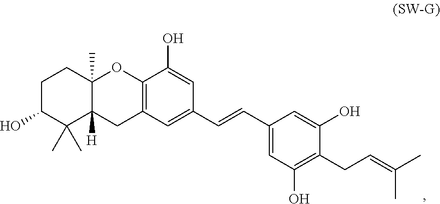 [SMILES: CC(C)=CCc1c(O)cc(/C=C/c2cc(O)c3c(c2)C[C@@H]2C(C)(C)[C@H](O)CC[C@@]2(C)O3)cc1O]

schweinfurthin K of formula (SW-K),

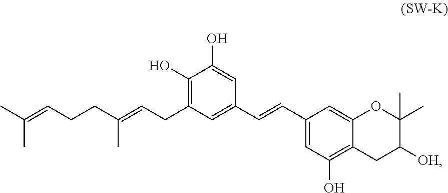 [SMILES: CC(C)=CCC/C(C)=C/Cc1cc(/C=C/c2cc(O)c3c(c2)OC(C)(C)C(O)C3)cc(O)c1O]

and schweinfurthin R of formula (SW-R)

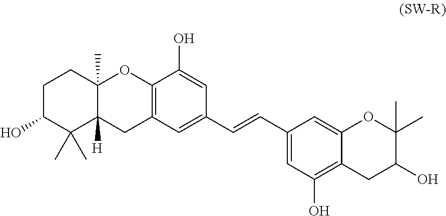 [SMILES: CC1(C)Oc2cc(/C=C/c3cc(O)c4c(c3)C[C@@H]3C(C)(C)[C@H](O)CC[C@@]3(C)O4)cc(O)c2CC1O]

characterized in that it comprises a step of using mappain of formula (IV)

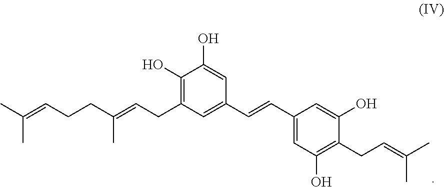 [SMILES: CC(C)=CCC/C(C)=C/Cc1cc(/C=C/c2cc(O)c(CC=C(C)C)c(O)c2)cc(O)c1O]

According to a second of its aspects, the present invention relates to schweinfurthin R (SW-R) as defined above.

According to a third of its aspects, the present invention relates to the use of a schweinfurthin G, K or R as obtained via a process according to the invention, as a synthetic intermediate for obtaining a schweinfurthin derivative, in particular chosen from vedelianin, schweinfurthin E and schweinfurthin F.

Surprisingly, the inventors found that it was possible to obtain schweinfurthins G, K and R from mappain. Mappain is represented in the following description by formula (IV) below.

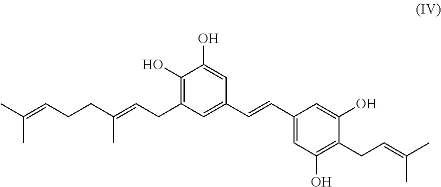 [SMILES: CC(C)=CCC/C(C)=C/Cc1cc(/C=C/c2cc(O)c(CC=C(C)C)c(O)c2)cc(O)c1O]

Schweinfurthin G is represented in the following description by the formula (SW-G) below.

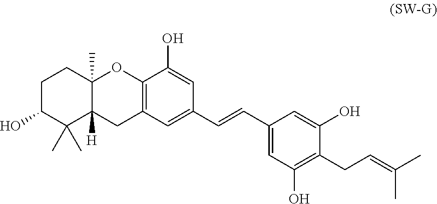 [SMILES: CC(C)=CCc1c(O)cc(/C=C/c2cc(O)c3c(c2)C[C@@H]2C(C)(C)[C@H](O)CC[C@@]2(C)O3)cc1O]

Schweinfurthin K is represented in the following description by the formula (SW-K) below.

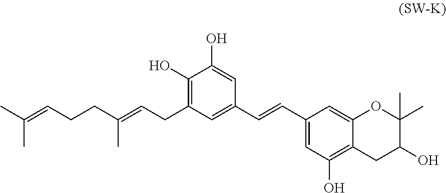 [SMILES: CC(C)=CCC/C(C)=C/Cc1cc(/C=C/c2cc(O)c3c(c2)OC(C)(C)C(O)C3)cc(O)c1O]

Schweinfurthin R is represented in the following description by the formula (SW-R) below.

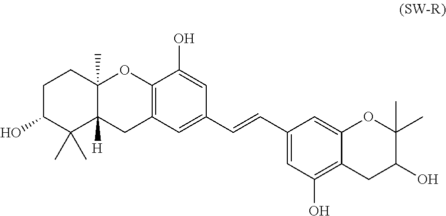 [SMILES: CC1(C)Oc2cc(/C=C/c3cc(O)c4c(c3)C[C@@H]3C(C)(C)[C@H](O)CC[C@@]3(C)O4)cc(O)c2CC1O]

According to a particular embodiment, mappain is obtained by extraction from plants of the genus Macaranga, in particular the leaves and fruits of Macaranga tanarius.

The present invention thus has the advantage of providing a process for the synthesis of schweinfurthins G, K and R from a renewable biobased precursor. It therefore allows considerable economic and ecological savings.

As may be seen from the examples given below, the synthetic route according to the present invention has a number of steps and a yield that cannot be attained by the total synthesis routes known in the prior art.

In addition, the facilitated access to schweinfurthins G, K and R makes it possible to envisage the future synthesis of novel schweinfurthin analogues for pharmacological study purposes.

Other characteristics, variants and advantages of a process according to the invention will emerge more clearly on reading the description and the examples that follow.

According to another of its aspects, the present invention relates to novel synthetic intermediates, represented hereinbelow by the formulae (I-R), (I-K), (II-G), (II-K) and (II-R).

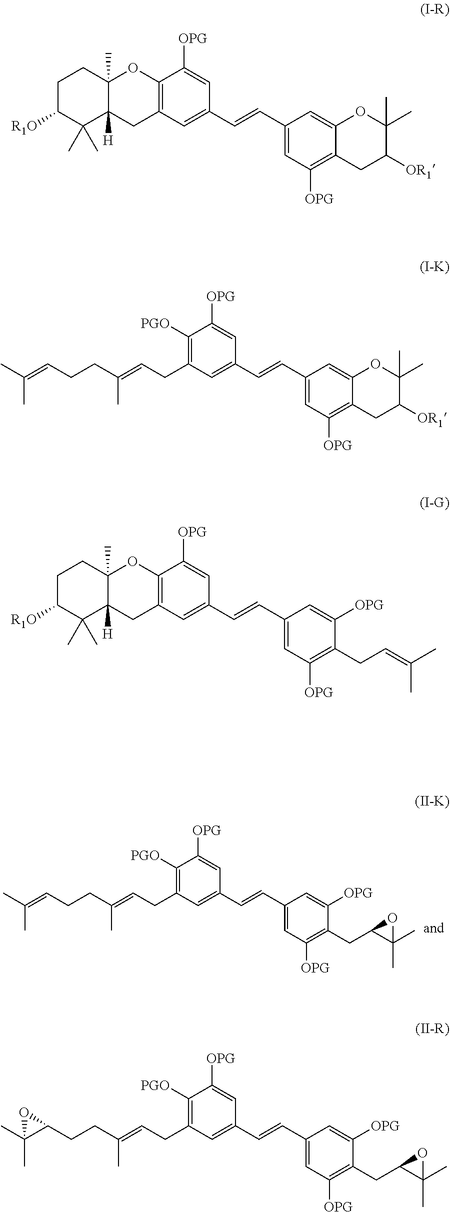

in which R₁ and R₁′ are independently chosen from the group consisting of hydrogen and PG, in which PG is a phenol-protecting group as defined below.

The present text also describes compounds (I-L), (I-S) and (II-T)

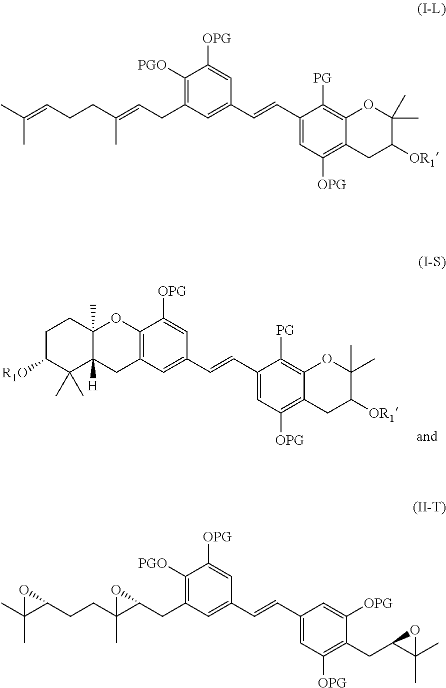

in which R₁ and R₁′ are independently chosen from the group consisting of hydrogen and PG, in which PG is a phenol-protecting group as defined below.

### BRIEF DESCRIPTION OF THE DRAWINGS

FIG. 1 represents a synthetic route for obtaining schweinfurthins G, K and R from the synthetic intermediates of formulae (II-G), (II-K) and (II-R) as defined above.

FIG. 2 represents a route for the synthesis of schweinfurthin G of formula (I) according to the invention.

Compounds (I-L), (I-S) and (II-T) are not shown in these figures.

### DETAILED DESCRIPTION

For the purposes of the present invention, the term “biobased” is intended to denote a compound extracted or isolated from a natural raw material. In the context of the present invention, plants of the genus Macaranga are used as raw materials.

In the context of the present invention, the use of the term “schweinfurthin derivative” is a sufficiently specific term for a person skilled in the art, not including an infinite number of structures, as is well reported in the publication Harmalkar Dipesch et al. RSC Adv. 2018, 8, 21191. At the present time, the schweinfurthin derivatives are well listed: there are 20 of them (Table 1, pages 21193-21195).

According to a particular embodiment, the process of the invention involves the implementation of at least four synthetic steps (i) to (iv) explained below.

According to this embodiment, seven synthetic intermediates (I-K), (I-G), (I-R), (II-R), (II-G), (II-K) and (III) defined above are obtained and the novel intermediates (I-K), (I-R), (II-R), (II-G) and (II-K) also form part of the invention.

In addition, according to this embodiment, the process according to the invention also allows the formation of the by-products (I-L), (I-S) and (II-T).

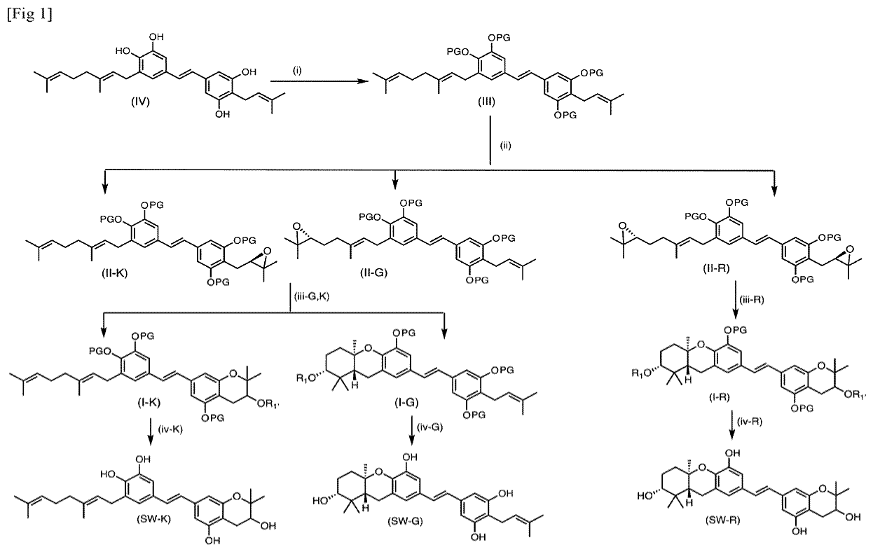

The scheme for the total synthesis leading from mappain of formula (IV) to the schweinfurthins G, K and R, respectively, of formulae (SW-G), (SW-K) and (SW-R) is illustrated in FIG. 1.

The synthesis of schweinfurthins G, K and R may be initiated by a mappain protection step according to scheme 1 below.

### Step (i)

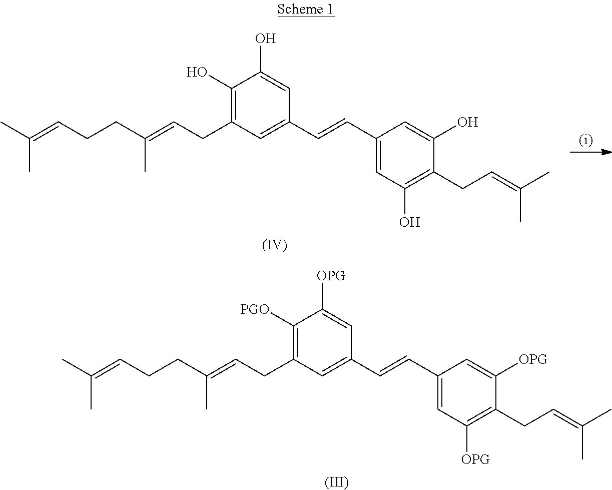

Step (i) as described in the synthetic scheme illustrated in FIG. 1 corresponds to the protection of mappain (of formula (IV)) to obtain the compound of formula (III) in which PG is a phenol-protecting group as defined below.

This mappain protection step can be performed by placing mappain in contact with a compound of formula PG-X in which X is a nucleofugal group chosen from halogens, tosylate, mesylate, nonaflate, phosphate, sulfamate or triflate, in particular chosen from halogens, tosylate, mesylate, nonaflate or sulfamate, more particularly chosen from halogens, tosylate or mesylate, and preferably X is a halogen, in particular chlorine, and in which PG is a phenol-protecting group as defined below, to obtain a compound of formula (III) as defined previously.

The placing of the mappain of formula (IV) in contact with the compound of formula PG-X is preceded by the placing of the mappain in contact with a base chosen from organic and mineral bases.

Mineral bases that may be mentioned include potassium carbonate (K₂CO₃), sodium hydride (NaH), potassium hydroxide (KOH), sodium hydroxide (NaOH) and caesium carbonate (Cs₂CO₃). Organic bases that may be mentioned include pyridine, N,N-diisopropylethylamine (DIPEA), and 4-dimethylaminopyridine (DMAP).

The base may be present in a mole ratio with mappain ranging, for example, from 5 to 15, typically in a mole ratio of 10. These compounds may be placed in a solvent such as acetonitrile, acetone, DMF, dichloromethane, pyridine and a mixture thereof, preferably acetonitrile at a temperature between, for example, 0 and 70° C., in particular between 2° and 30° C., typically a temperature of 25° C. Once the reaction is complete, the reaction medium may be placed at a temperature ranging from −10 to 5° C., in particular at 0° C., and a compound of formula PG-X is added to the reaction medium, in which PG and X are as defined previously. The compound of formula PG-X may be added in a mole ratio with mappain ranging, for example, from 5 to 15, in particular from 6 to 9, and preferably in a mole ratio of 8. The reaction medium may be placed under stirring for a period of time ranging, for example, from 3 hours to 5 days, in particular from 4 to 7 hours, and preferably for a period of 5 hours. On conclusion of the reaction, the compound of formula (III) may be isolated by means of techniques known to those skilled in the art, such as extraction, washing, filtration, vacuum evaporation and column chromatography.

For the purposes of the present invention, the term “phenol-protecting group” or “PG” refers to any group which, after reaction with at least one hydroxyl group borne by an aromatic ring, prevents any adverse reaction from taking place with said at least one hydroxyl group. A protecting group must be removable, so as to reform said at least one hydroxyl group, via a conventional chemical or enzymatic reaction. The protecting group used is not predominant and may include common compounds such as allyls, benzyls, acetyls, chloroacetyls, thiobenzyls, benzylidines, phenacyls, alkyls, alkoxyls, silyl ethers and any other group that is capable of being chemically bonded to at least one hydroxyl group and then of being selectively removed therefrom so as to reconstitute the at least one hydroxyl group via a chemical or enzymatic reaction under mild conditions, i.e. conditions that are compatible with the nature of the product bearing said at least one hydroxyl group. Acceptable protecting groups are known to those skilled in the art and are mentioned in T. W. Greene, Protecting Groups In Organic Synthesis; Wiley: New York, 1981 and also in the references cited herein.

It has been found that during cyclization steps such as step (iii), and in particular steps (iii-G,K) and (iii-R) described below, certain PG groups tend to migrate, notably via an aromatic electrophilic substitution alpha to the protected phenol function on which the cyclization is taking place, as reported in Topczewski, J. J. et al., J. Org. Chem. 2011, 76, 909-919. This migration is particularly observed when the PG(s) used are alkoxyalkyl, alkoxyalkoxyalkyl or alkoxyaryl groups.

In the context of the present invention, the protecting groups of the PG phenols may be chosen from alkoxyalkyl, alkoxyalkoxyalkyl, alkoxyaryl, alkyl, and trialkylsilyl groups and preferably may be chosen from alkoxyalkyl, and in particular are methoxymethyl groups.

In a particular embodiment, a phenol-protecting group PG for the purposes of the present invention is chosen from the group consisting of methoxymethyl, benzoxymethyl, tert-butyldimethylsilyl, acetate, methyl, and mixtures thereof.

For the purposes of the present invention, MOM represents a methoxymethyl group.

For the purposes of the present invention, the term “alkyl” means a linear, secondary or tertiary saturated monovalent hydrocarbon-based radical, in particular comprising from 1 to 6 carbon atoms, such as methyl, ethyl, propyl, n-propyl, isopropyl, butyl, isobutyl, sec-butyl, tert-butyl, pentyl, isopentyl, hexyl and isohexyl groups.

For the purposes of the present invention, the term “aryl” means a monovalent aromatic hydrocarbon-based radical, comprising, for example, from 5 to 7 carbon atoms, such as a phenyl.

For the purposes of the present invention, the term “alkoxy” means an —O-alkyl radical where the term alkyl is as defined above, such as methoxyl, ethoxyl, 1-propoxyl, 2-propoxyl, butoxyl, tert-butoxyl and pentoxyl radicals. Thus, the terms “alkoxyalkyl”, “alkoxyalkoxyarylkyl” and “alkoxyaryl” refer to -alkyl-O-alkyl, -alkyl-O-alkyl-O-alkyl and -aryl-O-alkyl radicals, respectively.

In a particular embodiment, several phenol-protecting groups PG may be used during the synthesis. In this case, the compounds of formulae (II-K), (II-G), (II-R), (II-T), (I-K), (I-L), (I-G), (I-R) and (I-S) as defined above are liable to bear different PGs. All these variations are considered to fall within the scope of the present invention.

In an even more particular embodiment, a single PG-X compound is used in the course of the process according to the invention. According to this even more particular embodiment, the compounds of formulae (II-K), (II-G), (II-R), (II-T), (I-K), (I-L), (I-G), (I-R) and (I-S) as defined above then bear an identical PG.

For the purposes of the present invention, the term “nucleofugal” refers to a charged or uncharged atom or group, which is capable of detaching from an atom borne by what is considered to be the main or residual part of a substrate during a specific reaction, taking the bonding electron pair with it. Common nucleofugal species are chosen from the group comprising mesylate, tosylate, triflate, nonaflate, sulfamate, phosphate and halogens.

According to the following scheme 2, it is possible to subject compound (III) obtained in step (i) to an epoxidation step, making it possible to obtain at least one mixture of the compounds of formulae (II-K) and (II-G), and also optionally the compound of formula (II-R) or even the compound of formula (II-T).

### Step (ii)

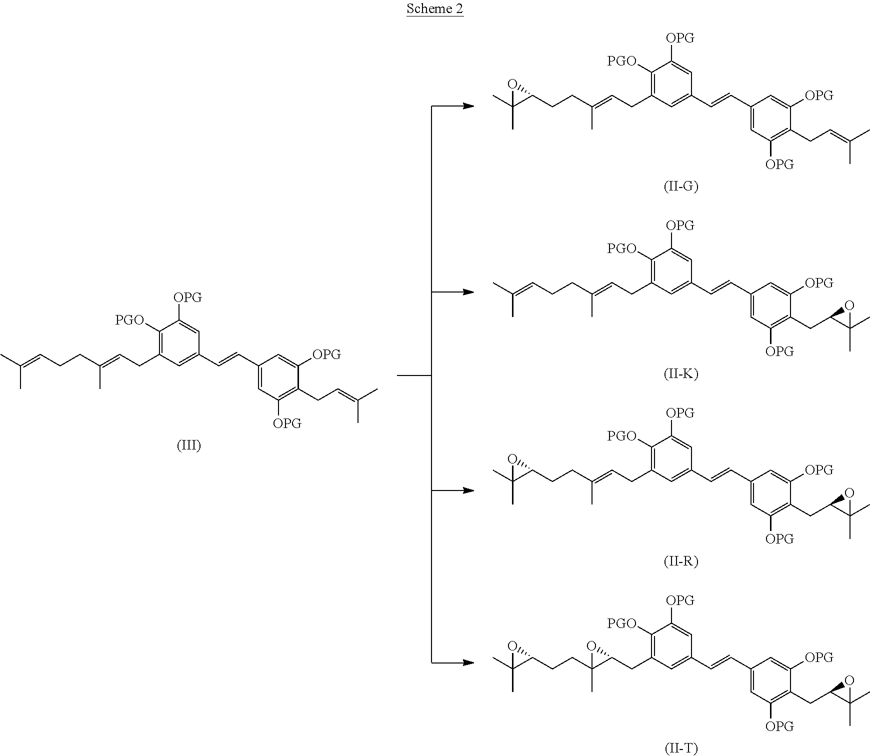

The object of the present invention is thus a process as described previously, characterized in that it comprises a step (ii) of epoxidation of a compound of formula (III) as defined previously in the presence of an oxidizing agent, notably a peroxide, and in particular hydrogen peroxide or potassium hydrogen persulfate, to obtain at least one compound chosen from the group consisting of the compounds of formulae (II-G) and (II-K) as defined previously, and optionally the compound of formula (II-R) as defined previously.

Thus, it will be developed in the following description that the composition of the reaction mixture obtained on conclusion of this epoxidation step is dependent on the synthetics conditions used.

Step (ii) as described in the synthetic scheme corresponds to the epoxidation of a compound of formula (III) to obtain a compound of formula (II-G) taken alone or as a mixture with at least one compound chosen from the compounds of formulae (II-K), (II-R) and (II-T), in particular as a mixture with the compounds of formulae (II-K), (II-R) and (II-T), where PG is a phenol-protecting group as defined previously.

Compounds (II-K) and (II-G) give the schweinfurthins K and G, respectively, whereas compound (II-R) can lead to schweinfurthin R. The description of these preparation methods will be developed hereinbelow. As regards compound (II-T), it is a by-product of step (ii). According to an alternative embodiment of the invention, compound (II-T) will not be involved in the process for synthesizing schweinfurthins G, K and R, for example by performing a separation step prior to performing the following step.

Depending on the reaction conditions of step (ii), compounds (II-K), (II-G), (II-R) and (II-T) may be obtained in different amounts. Conventionally, an asymmetric alkene epoxidation reaction without an alcohol in the allylic position may be performed in one step under the Jacobsen conditions (Zhang, W. et al. J. Am. Chem. Soc. 1990, 112, 2801-2803) in the presence of a manganese catalyst or under the Shi conditions (Wang, Z. X. et al. J. Am. Chem. Soc. 1997, 119, 11224-11235) in the presence of a chiral fructose-derived reagent, in particular under the Shi conditions.

In the case of a Jacobsen epoxidation, compound (III) may be placed in contact with a Jacobsen catalyst of formula (2)

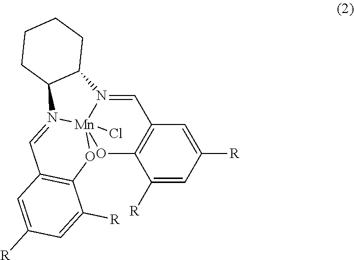

in which R is alkyl or alkoxy, R preferably being tert-butyl, and an oxidant such as sodium hypochlorite, N-methylmorpholine, or meta-chloroperbenzoic acid in a solvent such as dichloromethane, chloroform, dichloroethane, or ethyl acetate at a temperature of −5° C. to 30° C.

In the case of epoxidation under the Shi conditions, many parameters are liable to influence the reaction yield and the proportion of the compounds obtained.

According to a particular embodiment, the epoxidation is a Shi epoxidation, performed in the presence of a Shi reagent of formula (1)

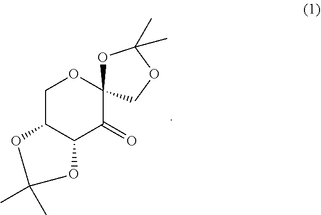

According to this particular embodiment, potassium hydrogen persulfate or hydrogen peroxide, advantageously hydrogen peroxide, may be mentioned as oxidant. The epoxidation may be performed in a solvent mixture (such as acetonitrile, dichloromethane, dimethyl ether, or a dichloromethane/acetonitrile/ethanol ternary mixture, preferably a dichloromethane/acetonitrile/ethanol ternary mixture) and an aqueous solution with a pH from 8 to 10.5, preferably from 8.5 to 9.5, for example at a temperature of 0 to 35° C., preferably at 20° C.

According to an even more particular embodiment, the oxidizing agent is present in a mole ratio relative to the compound of formula (III) ranging from 7 to 200, for example from 7 to 180, in particular from 13 to 16, and even more particularly this mole ratio being 15 or from 50 to 150, in particular from 80 to 120, and even more particularly this mole ratio being 100.

Typically, as illustrated in Example 1 below, in the case where the epoxidation is performed with hydrogen peroxide and with the Shi reagent of formula (1) as defined above, in a dichloromethane/acetonitrile/ethanol ternary mixture and an aqueous solution at pH 9.5, at room temperature, for example for 4 hours, a mole ratio between hydrogen peroxide and the compound of formula (III) as defined above of 15 makes it possible to form only the compounds of formulae (II-G) and (II-K) and the starting material (III).

Under conditions in which said mole ratio is higher, for example under conditions in which the mole ratio is 100, the pH is 9.5 and the reaction time is 50 hours, as illustrated in Example 2, the epoxidation generates the compounds (II-G), (II-K) and (II-R) and also the by-product (II-T).

In other words, for a pH of 9.5, a mole ratio between hydrogen peroxide and the compound of formula (III) ranging from 7 to 18 and for a reaction time of less than or equal to 6 hours, the compounds of formulae (II-G) and (II-K) are predominantly obtained. Whereas at a pH of 9.5, a mole ratio between hydrogen peroxide and the compound of formula (III) ranging from 50 to 150, in particular from 80 to 120, and even more particularly this mole ratio being 100, and a reaction time of greater than 24 hours, advantageously greater than or equal to 52 hours, a mixture of the compounds of formulae (II-G), (II-K) and (II-R) and also of the by-product of formula (II-T) is obtained.

According to one embodiment, it is possible to separate the compound of formula (II-R) and the by-product of formula (II-T) from the mixture of the compounds of formulae (II-G) and (II-K) by filtration and optionally by purification.

According to another embodiment, the compounds of formulae (II-G) and (II-K) are not separated before performing the next step. According to this particular embodiment, as shown in the description of step (ii′) below, compounds (II-T) and (II-R) are not formed.

Thus, depending on the mole ratios of oxidant and catalyst used relative to the amount of compound (III) and the reaction time, it is possible to obtain or not obtain compounds (II-R) and (II-T).

According to the preceding scheme 2, it is possible to subject compound (III) obtained in step (i) to an epoxidation step, making it possible to obtain at least one mixture of the compounds of formulae (II-K) and (II-G), and also optionally the compound of formula (II-R) or even the compound of formula (II-T).

The compound of formula (III) may be added to a reaction medium containing an oxidizing agent such as a peroxide, in particular hydrogen peroxide or potassium hydrogen persulfate, and a chiral fructose-based reagent such as the compound of formula (1) as defined above.

The chiral reagent may be present in a mole ratio with the compound of formula (III) ranging, for example, from 0.5 to 3, in particular from 1 to 2, and typically this mole ratio is from 1 to 1.3. The oxidizing agent may be present in a mole ratio with the compound of formula (III) ranging, for example, from 7 to 120. The reaction medium may be stabilized at a basic pH with the aid of a buffer solution, for example at a pH of from 8 to 11, in particular from 9 to 10 and typically the pH of the reaction medium is stabilized at a value of 9.5. The reaction medium may be placed under stirring for a period of time ranging, for example, from 2 hours to 2 days, at a temperature between, for example, 15 and 40° C., in particular between 20 and 30° C., and typically at a temperature of 25° C. Once the reaction is complete, the reaction medium may be placed at a temperature ranging from −10 to 5° C., in particular at 0° C.

Thus, according to a particular embodiment of the invention, the epoxidation of compound (III) leads to a mixture of compounds (II-G) and (II-K) according to scheme 3 below, in particular by implementing the operating conditions as described previously.

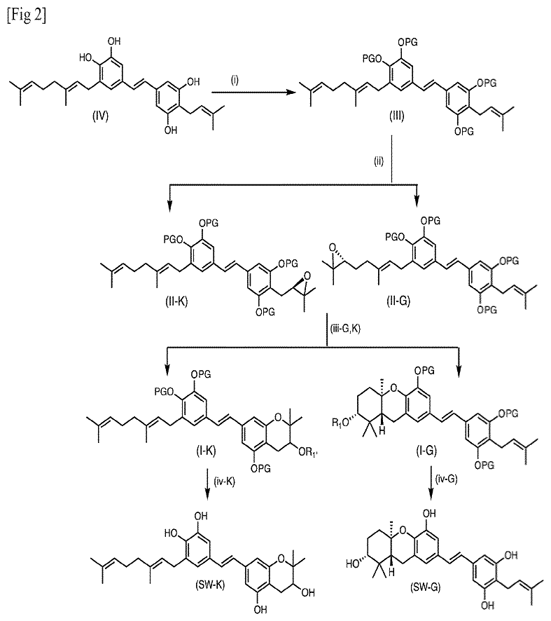

In this particular embodiment, the synthetic step (ii) corresponds to step (ii′) as illustrated in FIG. 2, more particularly dedicated to obtaining schweinfurthin G.

Step (ii′)

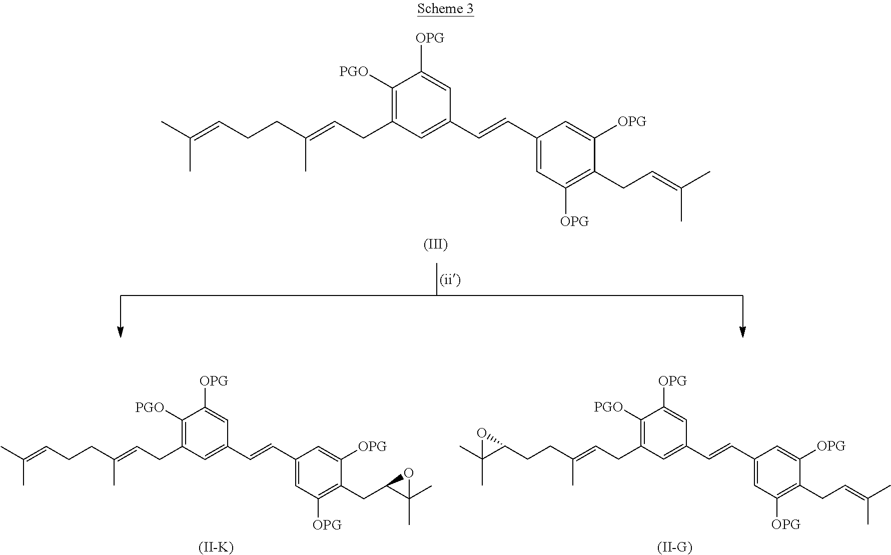

Step (ii′) as described in the synthetic scheme corresponds to the epoxidation of a compound of formula (III) to obtain a compound of formula (II-G) as a mixture with a compound of formula (II-K), in which PG is a phenol-protecting group as described previously.

The compound of formula (III) may be added to a reaction medium containing an oxidizing agent such as a peroxide, and in particular hydrogen peroxide or potassium hydrogen persulfate, and a chiral fructose-based reagent such as the compound of formula (1) as defined previously.

The chiral reagent may be present in a mole ratio with the compound of formula (III) ranging, for example, from 0.5 to 3, in particular from 1 to 2, and typically this mole ratio is from 1 to 1.3. The oxidizing agent may be present in a mole ratio with the compound of formula (III) ranging, for example, from 7 to 18, in particular from 13 to 16, and typically this mole ratio is 15. The reaction medium may be stabilized at a basic pH using a buffer solution, for example at a pH ranging from 8 to 11, in particular from 9 to 10, and typically the pH of the reaction medium is stabilized at 9.5. The reaction medium may be placed under stirring for a period of time ranging, for example, from 2 to 6 hours, in particular for 4 hours, at a temperature of, for example, 15 to 40° C., in particular between 20 and 30° C. and typically at a temperature of 25° C. Once the reaction is complete, the reaction medium may be placed at a temperature ranging from −10 to 5° C., in particular at 0° C.

The compounds obtained on conclusion of step (ii) are liable to be cyclized to form the schweinfurthins G, K and R in their protected forms (I-G), (I-K) and (I-R), respectively.

Step (iii)

According to a particular embodiment, step (iii) corresponds to step (iii-G, K), more particularly directed towards the synthesis of schweinfurthin G.

Step (iii-G,K)

According to scheme 4 below, step (iii-G, K) consists in reacting a mixture containing compounds (II-K) and (II-G) so as to obtain a mixture of compounds (I-K) and (I-G) and also the by-product of formula (I-L).

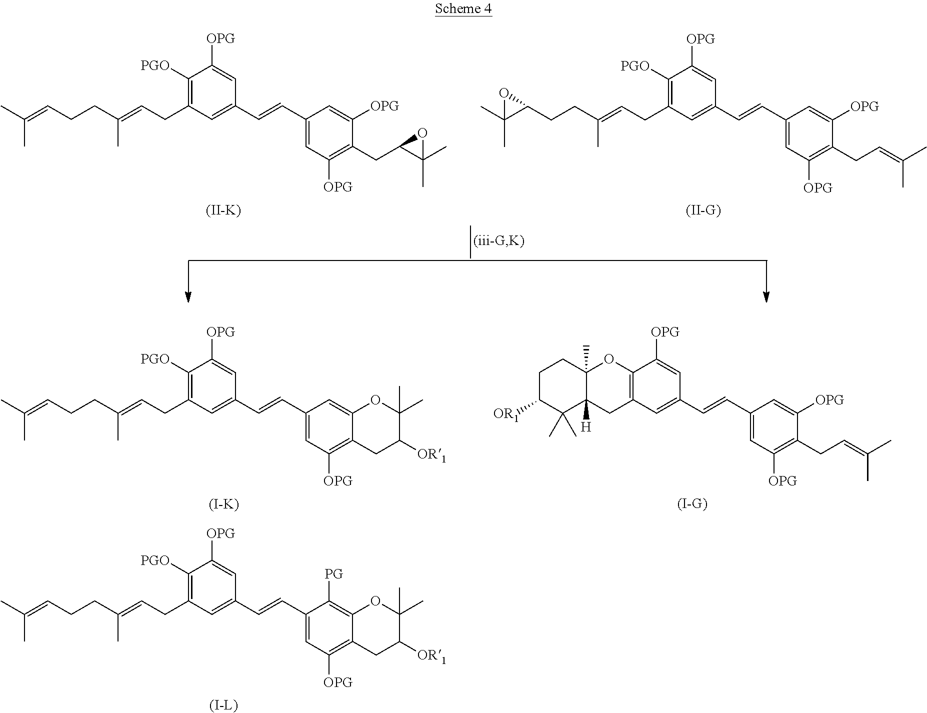

Step (iii-G,K) as described in the synthetic scheme of FIG. 2 is directed towards the cyclization of an epoxide of formula (II-G) as a mixture with the epoxide of formula (II-K) so as to obtain a compound of formula (I-G) and, where appropriate, a compound of formula (I-K) and also a by-product of formula (I-L) as defined above in which R₁ and R₁′ are independently chosen from the group consisting of a hydrogen atom and PG in which PG is a phenol-protecting group as described previously.

Indeed, it has been found that it is possible for the protecting group of the cyclized phenol to migrate onto the secondary alcohol formed at the time of cyclization or onto the aromatic ring.

According to a particular embodiment, the cyclization step (iii) is performed in the presence of a Lewis acid and of at least one compound chosen from the compounds of formulae (II-G) and (II-K) as defined previously to obtain the compound of formula (I-G), the compound of formula (I-K), the by-product of formula (I-L) as defined previously, or a mixture thereof, in which R₁ and R₁′ are independently chosen from the group consisting of a hydrogen and a PG group and in which PG is as defined previously.

A mixture containing the compounds of formulae (II-G) and (II-K) may be placed in a weakly polar solvent such as dichloromethane or a polar solvent such as hexafluoro-2-propanol (HFIP). The reaction medium may be placed at a temperature ranging, for example, from −100 to 20° C., in particular from −80 to 5° C., typically at a temperature of −78° C. or −10° C. A Lewis acid may be added to the reaction medium, for example in a mole ratio relative to the compounds of formulae (II-G) and (II-K) ranging from 0.5 to 8, in particular from 1 to 5, typically in a mole ratio from 1.5 to 4. The Lewis acid may be placed, for example, in an apolar aprotic solvent such as an alkane, in particular hexane. The reaction medium may be stirred at a temperature ranging, for example, from −100 to 20° C., in particular from −80 to 5° C., typically at a temperature of −78° C. or −10° C. for a period of time of, for example, 20 to 60 minutes, in particular for a period of 40 minutes. The reaction medium can be stabilized at room temperature after addition of a polar protic solvent such as water. It is possible to isolate the compounds of formulae (I-G) and (I-K) separately by filtration and optionally by purification.

In the context of the present invention, the term “Lewis acid” refers to any compound having an electron gap, capable of accepting an electron pair. Acceptable Lewis acids are known to those skilled in the art and are cited, for example, in the references cited herein: Lewis Acids in Organic Chemistry, 2000, volume 1, H. Yamamoto Ed., Wiley-VCH, and Avelino Corma, H. Garcia, Chem. Rev. 2003, 103, 4307-4365.

In the context of the present invention, the Lewis acid may be chosen from the group consisting of boron trifluoride sources, dialkylaluminium chlorides, tin chlorides, fluoro alcohols, and montmorillonite or metal trifluoromethanesulfonates and any other compound bearing an electron gap which is capable of accepting an electron pair, in particular boron trifluoride etherate, dimethylaluminium chloride, hexafluoroisopropanol, or zinc trifluoromethanesulfonate, and is preferably dimethylaluminium chloride.

According to a particular embodiment, the tetra-protected mappain of formula (III) remaining in the reaction medium on conclusion of step (iii-G,K) may be isolated and re-engaged in the preceding reaction sequence consisting of step (ii) and step (iii).

Compound (I-L), for its part, is a by-product of step (iii-G, K). According to an alternative embodiment of the invention, compound (I-L) will not be engaged in the process for synthesizing the schweinfurthins G and K, for example by performing a separation step prior to performing the next step.

According to another embodiment, step (iii) corresponds to step (iii-R) illustrated in FIG. 2, more particularly directed towards the synthesis of schweinfurthin R.

Step (iii-R)

According to scheme 5 below, step (iii-R) comprises cyclization of the compound of formula (II-R) to form the protected schweinfurthin (I-R) and also a by-product of formula (I-S).

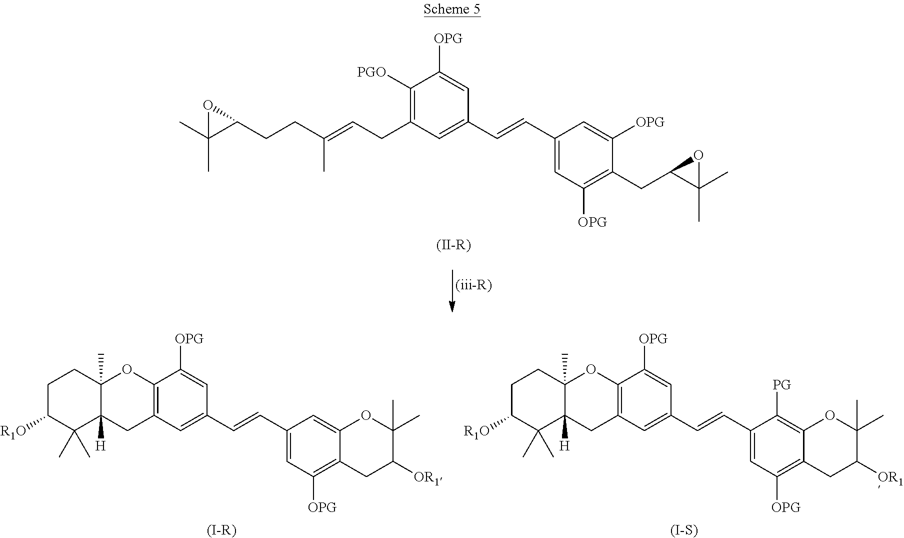

Step (iii-R) as described in the synthetic scheme is directed towards the cyclization of an epoxide of formula (II-R) taken alone or as a mixture with at least one of the epoxides of formulae (II-K), (II-R) and (II-T), more particularly taken alone, so as to obtain a compound of formula (I-R) as defined above and also a by-product of formula (I-S).

According to a particular embodiment, the cyclization step is performed in the presence of a Lewis acid and of a compound of formula (II-R) as defined previously to obtain the compound of formula (I-R) and the by-product of formula (I-S) as defined previously, in which PG, R1 and R₁′ are as defined previously.

Compound (I-S), for its part, is a by-product of step (iii-R). According to an alternative embodiment of the invention, compound (I-S) will not be engaged in the process for synthesizing schweinfurthin R, for example by performing a separation step prior to performing the next step.

As previously with respect to the PG groups, the compound of formula (I-R) may include identical or different groups R₁ and R₁′ and, as previously, the compound of formula (I-S) may be the result of a migration of the PG group from the cyclized phenol onto the secondary alcohol at the time of cyclization.

The compound of formula (II-R) may be placed in a weakly polar solvent such as dichloromethane or a polar solvent such as hexafluoro-2-propanol (HFIP). The reaction medium may be placed at a temperature ranging, for example, from −100 to 20° C., in particular from −80 to 5° C., typically at a temperature of −10° C. A Lewis acid may be added to the reaction medium, for example in a mole ratio relative to the compound of formula (II-R) ranging from 0.5 to 7, in particular from 1 to 5, typically in a mole ratio of 2.4. The Lewis acid may be placed, for example, in an apolar aprotic solvent such as an alkane, in particular hexane. The reaction medium may be stirred at a temperature ranging, for example, from −100 to 20° C., in particular from −80 to 5° C., typically at a temperature of −10° C. for a period of time of, for example, 20 to 150 minutes, in particular for a period of 90 minutes. The reaction medium can be stabilized at room temperature after addition of a polar protic solvent such as water. The compound of formulae (I-R) can be isolated by filtration and optionally purified.

According to scheme 6 below, the compounds obtained on conclusion of step (iii) are capable of undergoing deprotection in order to obtain the schweinfurthins G, K and R.

### Step (iv)

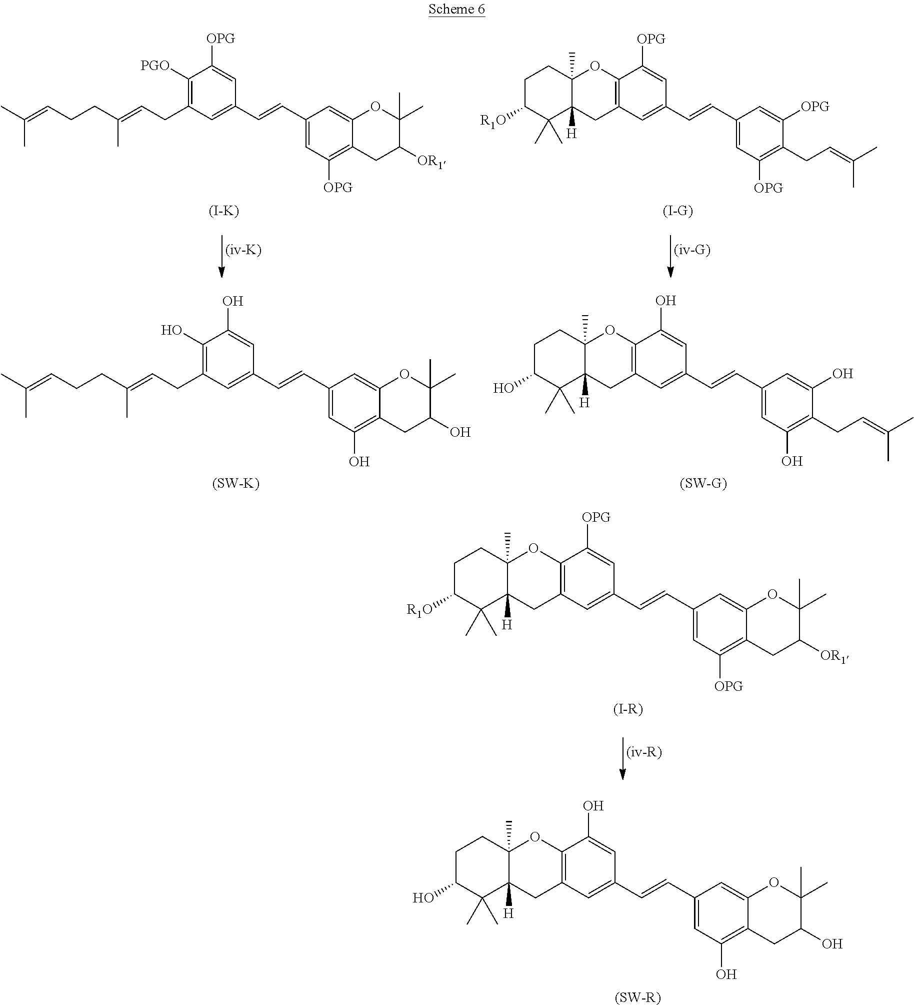

Step (iv) as described in the synthetic scheme corresponds to the deprotection of the compounds of formulae (I-G), (I-K) and (I-R), in which R₁ and R₁′ are independently chosen from the group consisting of hydrogen and PG, in which PG is a phenol-protecting group as described previously, to obtain the compounds of formulae (SW-G), (SW-K) and (SW-R), respectively.

According to a particular embodiment, the present invention is also directed towards a process for preparing schweinfurthin G of formula (SW-G) as defined previously, characterized in that it comprises a step (iv) of deprotection of a compound of formula (I-G) as defined previously.

According to another particular embodiment, the present invention is also directed towards a process for preparing schweinfurthin K of formula (SW-K) as defined previously, characterized in that it comprises a step (iv) of deprotection of a compound of formula (I-K) as defined previously.

According to another particular embodiment, the present invention is also directed towards a process for preparing schweinfurthin R of formula (SW-R) as defined above, characterized in that it comprises a step (iv) of deprotection of a compound of formula (I-R) as defined previously.

A compound chosen from the compounds of formulae (I-G), (I-K) and (I-R) may be placed in a polar protic solvent, for example an alcohol, in particular ethanol or isopropanol. A strong acid may be added to the reaction medium, for example a sulfonic acid, in particular optionally supported para-toluenesulfonic acid. The reaction medium may be heated to a temperature between, for example, 10 and 60° C., in particular between 20 and 50° C., typically at a temperature ranging from 25° C. to 40° C. for a time ranging, for example, from 10 to 90 hours, in particular from 15 to 80 hours, typically for a time of 24 to 76 hours. The compound chosen from the compounds of formulae (I-G), (I-K) and (I-R) can then be isolated from the reaction medium by purification.

### Extraction

Mappain may be extracted according to extraction techniques known to those skilled in the art; for example from plants of the genus Macaranga, in particular from the leaves and fruits of these plants. According to a particular embodiment, mappain is extracted from the dried leaves of Macaranga mappa or from the dried fruit of Macaranga tanarius. According to this embodiment, said dried fruit may be washed with a polar protic solvent such as ethanol or methanol to obtain a solution. Said solution may be evaporated, in particular under vacuum. A solvent such as diethyl ether, methyl tert-butyl ether or tetrahydrofuran may then be added to obtain a solution. Said solution can be decanted so as to collect the fraction containing mappain. This fraction may be concentrated, in particular under vacuum.

Typically, the mappain content of said fraction may be between 20% and 40%; in particular, the mappain content of said fraction is 30%.

Any purification method can then be performed, in particular column chromatography or liquid-liquid partition centrifugal extraction so as to obtain pure mappain.

In a particular embodiment, the extraction is performed by liquid-liquid partition centrifugal extraction.

Typically, the overall synthetic yield obtained by performing the steps reported in FIG. 1 is between 1% and 15%, in particular between 3% and 12%.

The process according to the present invention notably makes it possible to obtain schweinfurthin G in an improved yield and a biobased precursor that is accessible in suitable amounts.

The present invention moreover relates to a process according to the present invention, characterized in that it also comprises a step of preparing a pharmaceutical composition comprising a schweinfurthin chosen from the compounds of formulae (SW-G), (SW-K) and (SW-R) as defined previously and pharmaceutically acceptable excipients.

Thus, the invention more particularly relates to a process for preparing schweinfurthin G also comprising a step of preparing a pharmaceutical composition comprising schweinfurthin G of formula (SW-G) and pharmaceutically acceptable excipients.

Furthermore, the present invention also relates to the use of a compound obtained via a process according to the present invention, as a synthetic intermediate for obtaining a schweinfurthin derivative.

Indeed, the syntheses of vedelianin, schweinfurthin E and schweinfurthin G have been described, respectively, in Topczewski J. J, Wiemer D. F., Tetrahedron Lett., 2011 Apr. 6; 52 (14): 1628-1630, Topczewski J. J. et al., J. Org. Chem., 2009, 74, 6965-6972 and Mente N. R. et al., J. Org. Chem., 2008, 73, 7963-7970 and as such, a person skilled in the art is able, starting from a compound obtained according to the present invention, to obtain a schweinfurthin derivative such as vedelianin, schweinfurthin E and schweinfurthin F.

According to a particular embodiment, the present invention is directed towards the use of schweinfurthin G as obtained according to the process of the invention as a synthetic intermediate for obtaining a schweinfurthin G derivative, in particular vedelianin, schweinfurthin E and schweinfurthin F.

The examples that follow are intended to describe the invention by way of illustration and in a non-limiting manner.

### Example 1: Synthesis of Schweinfurthins G and K

### Extraction of Mappain

50 kg of dried Macaranga tanarius fruit are washed with 2×70 L of ethanol. The ethanolic solution is partially evaporated under vacuum, and 5 L of water and 5 L of diethyl ether are then added. After separation of the phases by settling, the diethyl ether fraction is concentrated under vacuum. This fraction contains about 30% of mappain which can be obtained pure by purification on a column of silica, by liquid-liquid partition centrifugal extraction or by any other purification method.

### Step (i): Protection

Mappain (11.14 mmol; 5.0 g) and potassium carbonate (112.29 mmol; 15.5 g; 10 molar equivalents) are placed in a dry flask and the assembly is placed under argon. Anhydrous acetonitrile (223 mL) is added and the resulting brown suspension is stirred at room temperature for 30 minutes. The assembly is placed at 0° C. (ice bath) and chloromethyl methyl ether (98.75 mmol; 7.5 mL; 8.9 molar equivalents) is added dropwise via a syringe pump over a period of 1 hour. The yellow suspension is stirred at 0° C. for 5 hours (reaction monitoring by TLC).

Water (20 ml) is added at 0° C. to the reaction medium and the whole is then stirred for 5 minutes at room temperature before being placed in a separating funnel. The reaction medium is extracted three times with methyl t-butyl ether (150 mL then 2×100 mL). The combined organic phases are washed with saturated NaCl solution, dried over magnesium sulfate, filtered and concentrated under reduced pressure. The crude reaction product is purified by column chromatography on silica (solid deposition, 9/1 to 1/9 heptane/ethyl acetate gradient) to give the desired tetra-protected mappain (4.55 g; 65% yield).

Step (ii′): Epoxidation

The Shi reagent (6.53 mmol; 1.69 g; 1.3 molar equivalents), a 2/1/1 mixture of dichloromethane/acetonitrile/ethanol (44 mL), a buffer solution at pH 9.5 (24 mL) and 30% hydrogen peroxide solution (12 mL; 15 molar equivalents) are placed in a round-bottomed flask. The tetra-protected mappain (6.28 mmol; 3.9 g) dissolved in 44 mL of a 2/1/1 dichloromethane/acetonitrile/ethanol mixture is added dropwise at room temperature. The pale yellow two-phase solution is stirred at room temperature for about 4 hours (monitored by TLC to avoid the formation of di-epoxidized compounds).

The flask containing the reaction medium is placed at 0° C. (ice bath) and 50 mL of saturated Na₂S₂O₃ solution are then added gently. The organic phase is separated out by settling, and the aqueous phase is extracted with 3×50 mL of ethyl acetate. The combined organic phases are washed with NaCl solution, dried over magnesium sulfate and concentrated under vacuum.

Step (iii): Cyclization

The crude reaction product obtained in step (ii) (which contains about 25% of the epoxide of formula (II-G), 25% of regioisomeric epoxide of formula (II-K) and 50% of unreacted tetra-protected mappain of formula (III)) is used directly in the next step. The preceding crude reaction product (4.9 g) is placed in a dry two-necked flask equipped with a mechanical stirrer. It is dissolved in anhydrous dichloromethane (460 mL) and placed at −78° C. under an argon atmosphere. A 1M solution of dimethylaluminium chloride in hexane (92.50 mmol; 23 mL; 4 molar equivalents) is added dropwise. The resulting orange solution is stirred at −78° C. for 40 minutes. Water (6.2 mL) is added and the reaction medium is allowed to warm to room temperature.

After 20 minutes of stirring (the solution is yellow), magnesium sulfate is added (28.24 g). The whole is filtered then the filtrate is concentrated under reduced pressure. The crude reaction product is purified by column chromatography on silica (solid deposit, ethyl acetate/heptane gradient) to give the tri-protected schweinfurthin G of formula (I-G) (368 mg), the tri-protected schweinfurthin K of formula (I-K) where R₁′ is hydrogen (189 mg), the tetra-protected schweinfurthin K of formula (I-K) where R₁′ is a methoxymethyl group (120 mg) and the by-product (I-L) and the tetra-protected mappain of formula (III) (2.0 g).

Said tetra-protected mappain can be re-engaged in the preceding reaction sequence (step (ii) and then step (iii)).

After two iterative cycles, the protected schweinfurthin G of formula (I-G) is obtained in a yield of 16% over two steps, and the tri- and tetra-protected schweinfurthins K (I-K) are obtained in a yield of 13% over two steps. This yield can be increased, after three iterative cycles, to 19% for the protected schweinfurthin G of formula (I-G) and 16% for the protected schweinfurthins K (I-K).

### Step (iv): Deprotection

To a solution of protected schweinfurthin G (I-G) (0.03 mmol; 16 mg) in ethanol (1.3 mL) is added, portionwise, supported para-toluenesulfonic acid (0.67 mmol; 268 mg; 25 molar equivalents). The reaction medium is stirred at room temperature for four days and then at 40° C. for 24 hours. After filtration and concentration of the solvent, schweinfurthin G is obtained pure in a yield of 96%.

The deprotection of schweinfurthin K is performed in a similar manner from the tri- and tetra-protected intermediates (I-K) in isopropanol, for 48 hours at 40° C. After reverse-phase purification (C18 column, gradient of water/acetonitrile with 1% formic acid), schweinfurthin K is obtained pure in a yield of 30%.

In conclusion, the total yield for the synthesis of schweinfurthin G from mappain is 12% for four steps. That of schweinfurthin K is 3% for four steps.

### Example 2: Synthesis of Schweinfurthin R from the Compound of Formula (III)

The compound of formula (III) is obtained under the same conditions as in Example 1.

### Step (ii): Epoxidation

The Shi reagent (4.03 mmol; 1 g; 1.0 molar equivalent), a 2/1/1 mixture of dichloromethane/acetonitrile/ethanol (29 mL), a buffer solution at pH 9.5 (11.5 mL) and 30% hydrogen peroxide solution (322.65 mmol; 32.4 mL; 80 molar equivalents) are placed in a round-bottomed flask. The tetra-protected mappain (III) (4.03 mmol; 2.5 g) dissolved in 29 mL of a 2/1/1 dichloromethane/acetonitrile/ethanol mixture is added dropwise at room temperature over a period of 1 hour. The pale yellow two-phase solution is stirred at room temperature for about 48 hours. 30% hydrogen peroxide solution (8.1 mL; 20 molar equivalents) is added and the two-phase mixture is stirred at room temperature for a further 4 hours.

The flask containing the reaction medium is placed at 0° C. (ice bath) and 18 mL of saturated Na₂S₂O₃ solution are then added gently. The organic phase is separated out by settling, and the aqueous phase is extracted with 3×50 mL of ethyl acetate. The combined organic phases are washed with NaCl solution, dried over magnesium sulfate and concentrated under vacuum. The crude reaction product is purified by column chromatography on silica (solid deposition, 9/1 to 0/1 heptane/ethyl acetate gradient) to give four products: an inseparable mixture of the two mono-epoxides (II-K) and (II-G) (770 mg, 30%), the di-epoxide (II-R) (702 mg, 27%), and the tri-epoxide by-product (II-T) (102 mg, 4%).

Step (iii-D): Cyclization

### Formation of Schweinfurthin R:

The di-epoxide compound (II-R) (702 mg, 1.07 mmol) is placed in a dry two-necked flask equipped with a mechanical stirrer. It is dissolved in anhydrous dichloromethane (63 mL) and placed at −10° C. under an argon atmosphere. A 1M solution of dimethylaluminium chloride in hexane (2.1 mmol; 2.1 mL; 2 molar equivalents) is added dropwise. The resulting orange solution is stirred at −10° C. for 20 min before addition of a further amount of 1M dimethylaluminium chloride solution in hexane (0.4 mmol; 0.4 mL; 0.4 molar equivalent). The reaction is stirred at −10° C. for 1 hour. Water (0.2 mL) and then magnesium sulfate are added. The whole is filtered then the filtrate is concentrated under reduced pressure. The crude reaction product is purified by column chromatography on silica (solid deposition, ethyl acetate/heptane gradient) to give four products: the di-protected schweinfurthin R (111 mg, 18%), the tri-protected schweinfurthins R (200 mg, 29%), the tetra-protected schweinfurthin R (54 mg, 8%) corresponding to the intermediate of formula (I-R), and the by-product of formula (I-S) (47 mg).

Step (iv-R): Deprotection

The intermediate (I-R) is deprotected under the same conditions as in step (iv) of Example 1 using isopropanol as solvent, at 40° C. for 48 hours. Schweinfurthin R is obtained pure after reverse-phase purification (C18 column, gradient of water/acetonitrile with 1% formic acid) in a yield of 17%.

The chemical structures and the spectroscopic data of some of the compounds of the invention are illustrated in Table 1 below.

TABLE 1

| | | Characterization (NMR and |
|---|---|---|
| Compound | Structure | mass spectrometry) |
| (SW-R) | 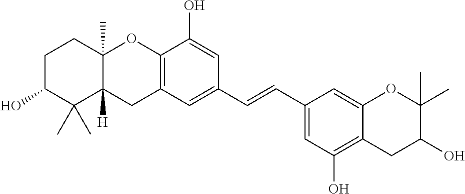 [SMILES: CC1(C)Oc2cc(/C=C/c3cc(O)c4c(c3)C[C@@H]3C(C)(C)[C@H](O)CC[C@@]3(C)O4)cc(O)c2CC1O] | ¹H NMR (CD₃CN, 500 MHz): δ (ppm) = 0.83 (s, 3H); 1.06 (s, 3H); 1.21 (s, 1H); 1.22 (s, 3H); 1.28 (s, 3H); 1.60 (m, 1H); 1.70 (m, 1H); 1.75 (m, 1H); 1.77 (m, 1H); 2.02 (m, 1H); 2.47 (dd, 1H, J = 6.8, 17.2 Hz); 2.72 (m, 2H); 2.83 (dd, 1H, J = 5.4, 17.2 Hz); 3.34 (m, 1H); 3.72 (m, 1H); 6.45 (d, 1H, J = 1.2 Hz); 6.52 (d, 1H, J = 1.2 Hz); |
| | | 6.81 (s, 1H); 6.81 (d, 1H, |
| | | J = 16.3 Hz); 6.85 (s, 1H); |
| | | 6.92 (d, 1H, J = 16.3 Hz); |
| | | RMN ¹³C (CD₃CN, 125 |
| | | MHZ): δ (ppm) = 14.8; 20.4; |
| | | 21.3; 23.6; 25.6; 27.1; 27.7; |
| | | 29.0; 38.5; 39.2; 48.2; 69.7; |
| | | 77.6; 78.0; 8.6; 105.2; 7107.3; |
| | | 108.1; 110.6; 120.5; 124.0; |
| | | 127.0; 129.1; 130.5; 138.3; |
| | | 141.7; 146.7; 155.8; 156.6; |
| | | HRMS (ESI): m/z calculated |
| | | for C₃₆H₃₇O₆⁺ [M + H]⁺: |
| | | 481,2590; found: 481,2562 |
| (I-R) R₁ = H R₁′ = H PG = MOM | 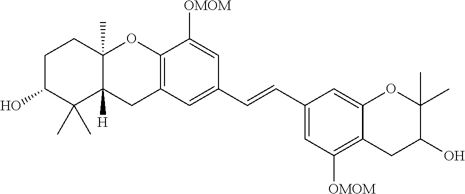 | RMN ¹H(CDCl₃, 300 MHz): δ (ppm) = 0.86 (s, 3H); 1.09 (s, 3H); 1.22 (s, 3H); 1.30 (s, 3H); 1.36 (s, 3); 1.58 (m, 1H); 1.68 (m, 1H); 1.78 (m, 1H); 1.82 (m, 1H); 2.09 (m, 1H); 2.72 (m, 3H); 2.92 (dd, 1H, J = 17.3, 5.3 Hz); 3.42 (m, 1H); 3.49 (s, 3H); 3.52 (s, 3H); 3.79 (m, 1H); 5.18 (m, 2H); 5.21 (s, 2H); 6.45 (d, 1H, J = 1.4 Hz); 6.75 (d, 1H, J = |
| | | 1.4 Hz); 6.79 (d, 1H, J = 16.1 |
| | | Hz); 6.90 (d, 1H, J = 1.8 Hz); |
| | | 6.91 (d, 1H, J = 16.1 Hz); 7.08 |
| | | (d, 1H, J = 1.8 Hz); |
| | | RMN ¹³C (CDCl₃, 75 MHz): |
| | | δ (ppm) = 14.6; 20.3; 22.6; |
| | | 23.5; 24.9; 27.0; 27.7; 28.4; |
| | | 38.1; 38.7; 47.2; 56.7 (2C); |
| | | 69.7; 77.1; 77.3; 78.4; 94.2; |
| | | 96.2; 104.3; 108.3; 108.7; |
| | | 114.0; 122.2; 123.5; 126.6; |
| | | 128.9; 129.5; 137.9; 144.5; |
| | | 148.6; 153.8; 156.7; |
| | | HRMS (ESI): m/z calculated |
| | | for C₃₃H₄₅NaO₈⁺ [M + H]⁺: |
| | | 569,3114; found: 569,3114 |
| (I-R) R₁ = H R₁′ = H PG = MOM | 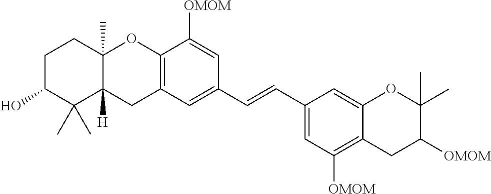 | ¹H NMR (CDCl₃, 300 MHz): δ (ppm) = 0.87 (s, 3H); 1.09 (s, 3H); 1.22 (s, 3H); 1.30 (s, 3H); 1.36 (s, 3H); 1.58 (m, 1H); 1.69 (m, 1H); 1.82 (m, 1H); 2.06 (m, 1H); 2.63 (m, 3H); 2.94 (dd, 1H, J = 17.2, 5.5 Hz); 3.40 (s, 3H); 3.43 (m, 1H); 3.48 (s, 3H); 3.52 (s, 3H); 3.72 (dd, 1H, J = 5.8, 6.8 Hz); 4.67 (d, 1H, J = 6.7 Hz); 4.81 (d, 1H, J = 6.7 Hz); 5.16 |
| | | (s, 2H); 5.21 (s, 2H); 6.66 (d, |
| | | 1H, J = 1.4 Hz); 6.73 (d, 1H, |
| | | J = 1.4 Hz); 6.88 (m, 2H); |
| | | 6.91 (d, 1H, J = 1.8 Hz); 7.08 |
| | | (d, 1H, J = 2.0 Hz); |
| | | RMN ¹³C (CDCl₃, 75 MHz): |
| | | δ (ppm) = 14.5; 20.0; 21.9; |
| | | 26.0; 27.6; 28.5; 23.7; 24.0; |
| | | 38.1; 38.7; 47.0; 55.9; 56.5 (2 |
| | | C); 74.5; 76.3; 77.0; 78.4; |
| | | 94.9 (2 C); 104.2;108.7; |
| | | 109.0; 114.6; 122.4; 123.5; |
| | | 126.7; 128.7; 128.9; 137.9; |
| | | 144.2; 146.4; 154.0; 156.0; |
| | | HRMS (ESI): m/z calculated |
| | | for C₃₅H₄₉O₉⁺ [M + H]⁺: |
| | | 613,3377; found: 613,3303 |
| (I-R) R₁ = MOM R₁′ = H PG = MOM | 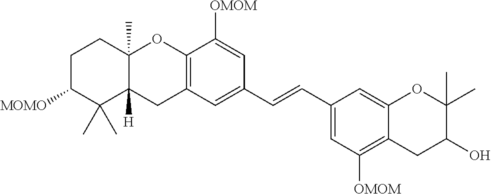 | ¹H NMR (CDCl₃, 300 MHZ): δ (ppm) = 0.90 (s, 3H); 1.09 (s, 3H); 1.25 (s, 3H); 1.32 (s, 3H); 1.37 (s, 3H); 1.58 (m, 1H); 1.72 (m, 1H); 1.78 (m, 1H); 1.98 (m, 1H); 2.09 (m, 1H); 2.72 (m, 2H); 2.73 (dd, 1H, J = 17.5, 5.1 Hz); 2.93 (dd, 1H, J = 17.5, 5.1 Hz); 3.29 (m, 1H); 3.41 (s, 3H); 3.51 (s, 3H); 3.53 (s, 3H); 3.81 (t, 1H, J = 4.9 Hz); 4.71 |
| | | (dd, 2H, J = 86.0, 8.2 Hz); |
| | | 5.20 (dd, 2H, J = 27.1, 6.4 |
| | | Hz); 5.23 (s, 2H); 6.68 (d, 1H, |
| | | J = 1.4 Hz); 6.76 (d, 1H, J = |
| | | 1.4 Hz); 6.88 (m, 2H); 6.91 |
| | | (d, 1H, J = 1.8 Hz); 7.10 (d, |
| | | 1H, J = 1.7 Hz); |
| | | RMN ¹³C (CDCl₃, 75 MHZ): |
| | | δ (ppm) = 15.3; 20.1; 22.4; |
| | | 23.4; 24.7; 25.4; 26.9; 27.5; |
| | | 37.8; 38.4; 47.2; 55.8; 56.3; |
| | | 56.4; 69.6; 76.8; 77.1; 84.2; |
| | | 94.6; 96.1; 96.3; 103.9; 108.2; |
| | | 108.6; 113.8; 122.4; 123.4; |
| | | 126.4; 128.6; 129.2; 137.7; |
| | | 144.2; 146.3; 153.7; 156.3; |
| | | HRMS (ESI): m/z calculated |
| | | for C₃₅H₄₉O₉⁺ [M + H]⁺: |
| | | 613,3377; found: 613,3371 |
| (I-R) R₁ = MOM R₁′ = MOM PG = MOM | 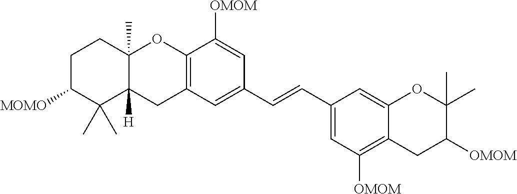 | ¹H NMR (CDCl₃, 300 MHz): δ (ppm) = 0.91 (s, 3H); 1.09 (s, 3H); 1.26 (s, 3H); 1.32 (s, 3H); 1.39 (s, 3H); 1.58 (m, 1H); 1.72 (m, 1H); 1.78 (m, 1H); 1.97 (m, 1H); 2.09 (m, 1H); 2.90 (m, 2H); 2.94 (dd, 1H, J = 17.2, 5.5 Hz); 3.28 (m, 1H); 3.41 (s, 3H); 3.42 (s, 3H); 3.51 (s, 3H); 3.52 (s, 3H); 3.76 (dd, 1H, J = 5.8, 6.8 Hz); 4.65 (d, 1H, J = |
| | | 8.2 Hz); 4.69 (d, 1H, J = 6.7 |
| | | Hz); 4.77 (d, 1H, J = 8.2 Hz); |
| | | 4.83 (d, 1H, J = 6.7 Hz); 5.19 |
| | | (s, 2H); 5.23 (s, 2H); 6.67 (d, |
| | | 1H, J = 1.4 Hz); 6.76 (d, 1H, |
| | | J = 1.4 Hz); 6.92 (m, 2H); |
| | | 6.95 (d, 1H, J = 1.8 Hz); 7.13 |
| | | (d, 1H, J = 1.7 Hz); |
| | | RMN ¹³C (CDCl₃, 75 MHz): |
| | | δ (ppm) = 15.3; 20.1; 26.0; |
| | | 23.4; 21.8; 25.4; 24.0; 27.5; |
| | | 37.8; 38.4; 47.2; 55.9; 56.0; |
| | | 56.4 (2C); 74.7; 76.2; 77.1; |
| | | 84.2; 94.6; 94.9; 95.6; 96.3; |
| | | 103.9; 108.6; 108.8; 111.8; |
| | | 114.8; 123.4; 126.6; 128.4; |
| | | 128.8; 138.0; 144.2; 146.3; |
| | | 155.9; 156.9; |
| | | HRMS (ESI): m/z calculated |
| | | for C₃₇H₅₃O₁₀⁺ [M + H]⁺: |
| | | 657,3633; found: 657,3618 |
| (I-S) R₁ = H R₁′ = H PG = MOM | 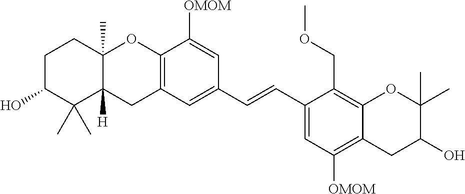 | ¹H NMR (CDCl₃, 300 MHZ): δ (ppm) = 0.87 (s, 3H); 1.09 (s, 3H); 1.22 (s, 3H); 1.31 (s, 3H); 1.35 (s, 3H); 1.60 (m, 1H); 1.71 (m, 1H); 1.79 (m, 1H); 1.83 (m, 1H); 2.07 (m, 1H); 2.71 (m, 3H); 2.93 (dt, 1H, J = 17.8, 5.0 Hz); 3.36 (s, 3H); 3.42 (m, 1H); 3.48 (s, 3H); 3.52 (s, 3H); 3.79 (t, 1H, J = 5.0 Hz); 4.59 (s, 2H); 5.17 (m, 2H); 5.21 (s, 2H); 6.85 (d, |
| | | 1H, J = 16.0 Hz); 6.89 (s, 1H); |
| | | 6.98 (d, 1H, J = 1.7 Hz); 7.09 |
| | | (d, 1H, J = 1.7 Hz); 7.26 (d, |
| | | 1H, J = 16.0 Hz); |
| | | RMN ¹³C (CDCl₃, 75 MHZ): |
| | | δ (ppm) = 14.6; 20.2; 22.4; |
| | | 23.5; 24.9; 27.0; 27.5; |
| | | 28.5; 37.9; 38.6; 47.0; 56.4 |
| | | (2C); 57.7; 64.6; 69.5; 76.9; |
| | | 77.1; 78.3; 94.5; 96.3; 102.9; |
| | | 108.3; 114.3; 122.1; 124.4; |
| | | 123.1; 129.8; 130.6; 138.4; |
| | | 144.3; 146.3; 152.6; 156.2; |
| | | HRMS (ESI): m/z calculated |
| | | for C₃₅H₄₈O₉Na⁺ [M + Na]⁺: |
| | | 635.3196; found: 635.3211 |
| (I-K) R₁′ = H PG = MOM | 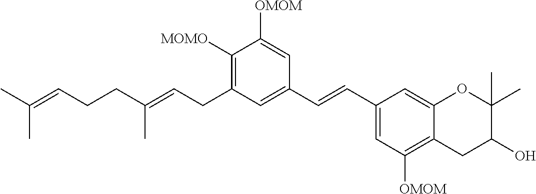 | ¹H NMR (CDCl₃, 500 MHz): δ (ppm) = 1.32 (s, 3H); 1.37 (s, 3H); 1.61 (s, 3H); 1.68 (s, 3H); 1.74 (s, 3H); 2.06 (m, 2H); 2.11 (m, 2H); 2.73 (dd, 1H, J = 16.7, 5.9 Hz); 2.93 (dd, 1H, H1″, J = 16.7, 5.9 (Hz); 3.42 (d, 2H, J = 7.9 Hz); 3.51 (s, 3H); 3.53 (s, 3 H); 3.60 (s, 3 H); 3.81 (t, 1H, J = 5.0 Hz); 5.12 (s, 3 H); 5.23 (s, 2H); 5.24 (s, 2H); 5.34 (t, 1 H, J = 6.3 Hz); 6.70 (s, 1H); 6.78 |
| | | (s, 1 H); 6.91 (m, 2 H); 6.95 |
| | | (d, 1H, J = 2.0 Hz); 7.14 |
| | | (d, 1H, J = 2.0 Hz); |
| | | RMN ¹³C (CDCl₃, 125 MHZ): |
| | | δ (ppm) = 16.4; 17.9; 22.3; |
| | | 24.7; 25.9; 26.9 (2C); 28.6; |
| | | 39.9; 56.3; 56.4; 57.7; 69.6; |
| | | 94.6; 95.4; 99.3; 104.0; 108.5; |
| | | 108.9; 111.9; 121.9; 122,; |
| | | 124.4; 128.9 6; 128.6; 131.6; |
| | | 133.7; 136.3; 136.6; 137.4; |
| | | 143.8; 150.1; 153.7 156.3; |
| | | HRMS (ESI): m/z calculated |
| | | for C₃₅H₄₉O₈⁺ [M + H]⁺: |
| | | 597.3427; found: 597.3465 |
| (I-K) R₁′ = MOM PG = MOM | 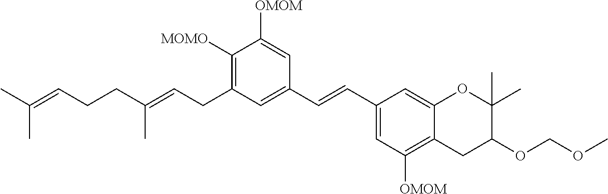 | ¹H NMR (CDCl₃, 500 MHz): δ (ppm) = 1.28 (s, 3 H); 1.35 (s, 3 H); 1.58 (s, 3H); 1.65 (s, 3 H); 1.71 (s, 3H); 2.07 (m, 4H); 2.66 (dd, 1 H, J = 17.4, 7.0 Hz); 2.95 (dd, 1 H, J = 17.4, 5.1 Hz); 3.40 (s, 3H); 3.41 (m, 2 H); 3.48 (s, 3 H); 3.51 (s, 3 H); 3.58 (s, 3 H); 3.74 (dd, 1H, J = 7.1, 5.1 Hz); 4.67 (d, 1H, J = 7.0 Hz); 4.81 (d, 1H, J = 6.9 Hz); 5.10 |
| | | (s, 3 H); 5.22 (s, 4 H); 5.32 (t, |
| | | 1 H, J = 6.3 Hz); 6.81 (s, 1H); |
| | | 6.85 (s, 1 H); 6.91 (m, 2 H); |
| | | 6.95 (d, 1H; J = 2.0 Hz); 7.11 |
| | | (d, 1H; J = 2.0 Hz); |
| | | RMN ¹³C (CDCl₃, 125 MHz): |
| | | δ (ppm) = 16.4; 18.0; 21.5; |
| | | 24.0; 25.1; 25.9; 26.9; 28.7; |
| | | 39.9; 56.4 (2 C); 57.8; 74.9; |
| | | 95.8; 95.0 (2 C); 99.4; 104.3; |
| | | 105.8; 109.1; 112.0; 122.0; |
| | | 122.8; 124.6; 128.5 (2 C); |
| | | 133.7; 131.8; 133.7; 136.5 |
| | | (2 C); 137.4; 143.8; 150.1; |
| | | 153.8; 156.4; |
| | | HRMS (ESI): m/z calculated |
| | | for C₃₇H₅₂NaO₉⁺ [M + Na]⁺: |
| | | 663.3509; found: 663.3505 |
| (I-L) R_{1′} = H PG = MOM | 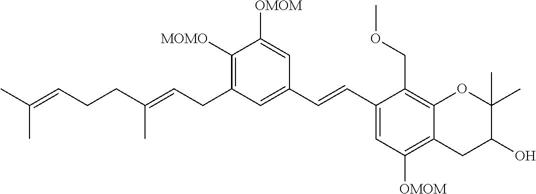 | ¹H NMR (CDCl₃, 500 MHz); δ (ppm) = 1.30 (s, 3 H); 1.35 (s, 3 H); 1.57 (s, 3H); 1.65 (s, 3 H); 1.71 (s, 3H); 2.05 (m, 4H); 2.72 (dd, 1 H, J = 17.7, 5.3 (Hz); 2.93 (dd, 1 H, J = 17.7, 5.3 Hz); 3.36 (s, 3 H); 3.42 (d, 2 H, J = 7.2 Hz); 3.48 (s, 3 H); 3.51 (s, 3 H); 3.58 (s, 3 H); 3.79 (t, 1H, J = 5.3 Hz); 4.56 (d A-B, 2 H, J = 11.6 Hz); 5.09 (m, 1 H); 5.10 (s, 2 |
| | | H); 5.21 (s, 2 H); 5.22 (s, 2 |
| | | H); 5.32 (t, 1 H, J = 7.1 Hz); |
| | | 6.88 (d, 1 H, J = 1.9 Hz); |
| | | 6.89 (s, 1 H); 6.99 (d, 1 H, |
| | | J = 1.9 Hz); 7.13 (d, 1 H, J = |
| | | 1.9 Hz); 7.29 (d, 1 H, J = 16.2 |
| | | Hz); |
| | | RMN ¹³C (CDCl₃, 125 MHz); |
| | | δ (ppm) = 16.4; 18.1; 22.4; |
| | | 24.7; 25.9; 26.9; 27.1; 28.7; |
| | | 40.2; 56.4 (2 C); 57.7; 58.1; |
| | | 64.8; 69.4; 94.8; 95.2; 99.4; |
| | | 102.9; 108.4; 112.6; 117.74; |
| | | 122.0; 122.9; 124.5; 126.1; |
| | | 130.5; 131.8; 134.9; 136.8 |
| | | (2 C); 138.3; 144.6; 150.6; |
| | | 152.1; 156.2; |
| | | HRMS (ESI): m/z calculated |
| | | for C₃₇H₅₂NaO₉⁺ [M+Na]⁺: |
| | | 663.3509; found: 663.3568 |
| (I-G) R₁ = H PG = MOM | 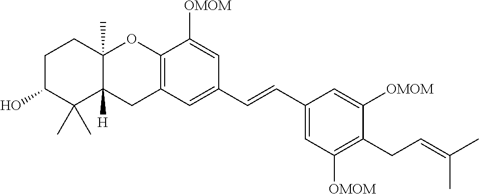 | ¹H NMR (CDCl₃, 300 MHz): δ (ppm) = 0.89 (s, 3 H); 1.11 (s, 3 H); 1.25 (s, 3H); 1.66 (s, 3H); 1.76-1.69 (m, 1H); 1.79 (s, 3 H); 1.91-1.81 (m, 2 H); 2.14-2.06 (m, 1 H); 2.71 (d, 2 H, J = 3.3 Hz); 2.74 (s, 1 H, OH); 3.39 (d, 2 H, J = 7.7 Hz); 3.43 (s, 1 H); 3.50 (s, 6 H); 3.54 (s, 3 H); 5.24-5.17 (m, 3 H); 5.23 (s, 4 H); 6.91-6.87 (m, 4 H); 6.96 (d, 1 H, J = 1.9 |
| | | Hz); 7.12 (d, 1H, J = 1.9 Hz); |
| | | RMN ¹³C (CDCl₃, 75 MHz): δ |
| | | (ppm) = 14.4; 17.9; 20.1; |
| | | 22.9; 23.4; 25.9; 27.5; |
| | | 28.5; 37.9; 38.6; 47.0; 56.1 (2 |
| | | C); 56.4; 77.2; 78.2; 94.7 (2 |
| | | C); 96.1; 106.2 (2 C); 113.9; |
| | | 119.7; 122.3; 121.9; 123.0; |
| | | 123.3, 126.8; 128.2; 129.3; |
| | | 131.1; 136.9; 144.1; 146.3; |
| | | 156.0 (2C); |
| | | HRMS (ESI): m/z calculated |
| | | for C₃₅H₄₉O₈⁺ [M + H]⁺: |
| | | 597.3422; found: 597.3425 |
| (II-K) PG = MOM | 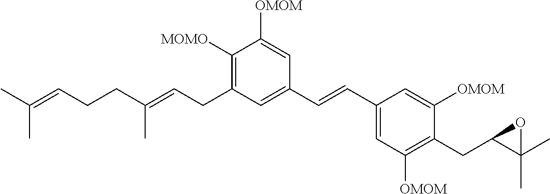 | ¹H NMR (CDCl₃, 500 MHZ): δ (ppm) = 1.26 (s, 3H); 1.41 (s,3H); 1.58 (s, 3H); 1.66 (s, 3H); 1.73 (s, 3H); 2.05 (m, 2H); 2.10 (m, 2H); 2.81 (dd, 1H, J =13.5, 7.5 Hz); 2.96 (dd, 1H, J = 7.3, 4.3 Hz); 3.09 (dd, 1H, J =13.5, 4.5 Hz); 3.42 (d, 2H, J = 7.0 Hz); 3.49 (s, 6H); 5.09 (m, 1H); 3.52 (s, 3H); 3.59 (s, 3H); 5.10 (s, 2H); 5.22 (s, 6H); 5.32 (t, 2H, J = |
| | | 7.0 Hz); 6.90 (m, 1H); 6.93 (s, |
| | | 2H); 6.96 (s, 1H); 6.97 (m, |
| | | 1H); 7.14 (s, 1H); |
| | | RMN ¹³C (CDCl₃, 125 MHz): |
| | | δ (ppm) = 16.3; 18.0; 19.4; |
| | | 23.8; 25.15; 25.9; 27.0; 28.8; |
| | | 40.0; 56.2 (2 C); 56.6; 57.8; |
| | | 59.3; 64.0; 95.4 (3 C); 99.6; |
| | | 106.2 (2 C); 112.0; 116.0; |
| | | 122.1; 123.0; 124.5; 128.1; |
| | | 128.8; 131.8; 133.7 (2 C); |
| | | 136.5; 137.7; 144.7; 150.1; |
| | | 156.9 (2 C): |
| | | HRMS (ESI): m/z calculated |
| | | for C₃₇H₅₂O₉Na+ [M + Na]⁺: |
| | | 663.3003; found: 663.3510 |
| (II-G) PG = MOM | 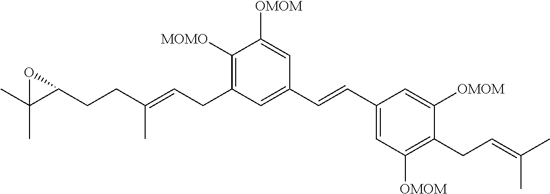 | ¹H NMR (CDCl₃, 500 MHz): δ (ppm) = 1.24 (s, 3H); 1.25 (s, 3H); 1.65 (s, 3H); 1.65 (m, 2H); 1.75 (m, 3H); 1.77 (s, 3H); 2.70 (t, 1H, J = 6.2 Hz); 3.37 (d, 2H, J = 7.0 Hz); 3.42 (d, 2H, J = 7.0 Hz); 3.48 (s, 6H); 3.51 (s, 3H); 3.58 (s, 3H); 5.10 (s, 2H); 5.18 (m, 1H); 5.22 (s, 6H); 5.38 (5, 1H, J = 7.0 Hz); 6.90 (m, 1H); 6.89 (s. 2H); 6.94 (s, 1H); |
| | | 6.93 (m); 7.13 (s, 1H); |
| | | RMN ¹³C (CDCl₃, 125 MHz): |
| | | δ (ppm) = 16.3; 18.0; 19.1; |
| | | 23.2; 25.1; 26.0; 27.6; 28.9; |
| | | 36.6; 56.2 (2 C); 56.6; 57.8; |
| | | 58.4; 64.6; 94.8 (3); 99.6; |
| | | 106.4 (2 C); 112.1; 120.1; |
| | | 122.9; 120.0; 123.5; 128.1; |
| | | 128.4; 131.3; 133.8 (2 C); |
| | | 135.6; 137.7; 144.6; 150.1; |
| | | 156.3 (2 C); |
| | | HRMS (ESI): m/z calculated |
| | | for C₃₇H₅₂O₉Na⁺ [M + Na]⁺: |
| | | 663.3503; found: 663.3510 |
| (II-R) PG = MOM | 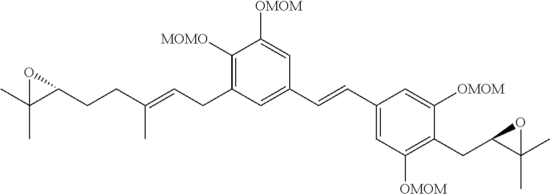 | ¹H NMR (CDCl₃, 500 MHz): δ (ppm) = 1.27 (s, 3 H); 1.28 (s, 3H); 1.29 (s, 3H, H 5″); 1.44 (s, 3 H); 1.75-1.62 (m, 2 H); 1.78 (s, 3H); 2.29-2.14 (m, 2 H); 2.74 (t, 1 H, J = 6.3 Hz); 2.84 (dd, 1 H, J = 13.2, 7.2 Hz); 3.03-2.96 (m, 1 H); 3.12 (dd, 1 H, J = 13.2, 4.4 Hz); 3.45 (d, 2 H, J = 7.4 Hz); 3.53 (s, 6 H); 3.55 (s, 3 H); 3.61 (s, 3 H); 5.14 (s, 2 H); 5.25 (s, 2 |
| | | H); 5.26 (s, 4 H); 5.41 (t, 1 H, |
| | | J = 7.4 Hz); 6.97-6.87 (m, 4 |
| | | H) 6.99 (d, 1 H, J = 1.8 Hz); |
| | | 7.17 (d, 1H, H₃, J = 1.8 Hz); |
| | | RMN ¹³C (CDCl₃, 125 MHz): |
| | | δ (ppm) = 16.4; 18.9; 19.2; |
| | | 23.6; 25.0; 25.1; 27.6; 28.8; |
| | | 36.6; 56.3 (2C); 56.4; 57.7; |
| | | 58.5; 59.2; 63.9; 64.3; 94.7 |
| | | (2 C); 95.3;99.3; 106.1 (2 C); |
| | | 112.0; 115.9; 121.9; 123.4; |
| | | 128.1; 128.6; 133.7; 135.6; |
| | | 136.1; 137.6; 144.7; 150.2; |
| | | 156.6 (2 C); |
| | | HRMS (ESI): m/z calculated |
| | | for C₃₇H₅₃O₁₀⁺ [M + H]⁺: |
| | | 657.3633; found: 657.3638 |
| (II-T) PG = MOM | 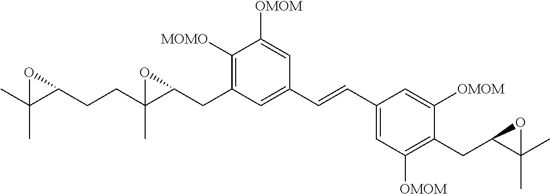 | RMN ¹H (CDCl₃, 500 MHz): δ (ppm) = 1.24 (m, 9H); 1.53 (s, 6H); 1.72-1.57 (m, 4H); 2.66 (t, 1 H, J = 6.1 Hz); 2.83 (m, 1 H); 2.96 (m, 1H); 2.97 (m, 2H); 3.07 (m, 1 H); 3.10 (m, 1 H); 3.49 (s, 6 H); 3.52 (s, 3 H); 3.57 (s, 3 H); 5.13 (s, 2 H); 5.23 (s, 6 H); 6.99-6.87 (m, 4 H); 7.06 (d, 1 H, J = 1.8 Hz); 7.19 (d, 1H, J = 1.8 Hz); 7.19 (d. 1H, J = 1.8 Hz); |
| | | RMN ¹³C (CDCl₃, 125 MHZ): |
| | | δ (ppm) = 17.1; 18.5; 19.3 (2 |
| | | C); 24.1; 25.2 (3C); 30.1; |
| | | 35.7; 56.8 (3C); 57.9; 59.0; |
| | | 62.4; 62.6; 64.2 (3 C); 95.2 (3 |
| | | C); 99.9; 106.3 (2 C); 113.1; |
| | | 115.9; 122.7; 128.4 (2 C) |
| | | 132.4 (2 C); 133.9; 137.3; |
| | | 144.8 (2 C); 156.5 (2 C) |
| | | HRMS (ESI): m/z calculated |
| | | for C₃₇H₅₃O₁₁⁺ [M + H]⁺: |
| | | 673.3582; found: 674.3542 |
| (III) PG = MOM | 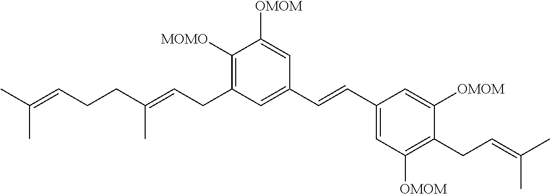 | ¹H NMR (CDCl₃, 500 MHz): δ (ppm) = 1.61 (s, 3H); 1.67 (s, 3 H); 1.68 s, 3 H); 1.75 (s, 3H); 1.79 (s, 3 H); 2.09 (m, 2 H); 2.12 (m, 2 H); 3.39 (d, 2 H, J = 7.2 Hz); 3.44 (d, 2 H, J = 7.2 Hz); 3.51 (s, 6 H); 3.54 (s, 3 H); 3.61 (s, 3 H); 5.12 (s, 3 H); 5.21 (t, 1 H , J = 7.1 Hz); 5.24 (s, 6H); 5.35 (t, 1 H, J = 6.8 Hz); 6.91 (s, 2 H); 6.92 (s, 1H); 6.93 (s, 1H); 6.98 (d, |
| | | 1 H, J = 1.8 H); 7.16 (d, 1H, |
| | | J = 1.9 Hz); |
| | | RMN ¹³C (CDCl₃, 125 MHZ): |
| | | δ (ppm) = 16.4; 17.9; 17.9, |
| | | 23.0; 25.9; 26.0; 26.8; 28.6; |
| | | 39.9; 56.2 (2 C); 56.4; 57.6; |
| | | 94.6 (2 C); 95.3; 99.2; 106.2 |
| | | (2 C); 111.7; 119.9; 121.9; |
| | | 122.7; 122.9; 124.4; 128.1; |
| | | 128.2; 131.2; 131.5; 133.7; |
| | | 136.3; 136.4; 136.6; 144.5; |
| | | 150.1; 156.0 (2 C); |
| | | HRMS (ESI): m/z calculated |
| | | for C₃₇H₅₃O₈⁺ [M + H]⁺: |
| | | 625.37350; found: 625.3748 |

1. Process for preparing at least one schweinfurthin chosen from schweinfurthin G of formula (SW-G), 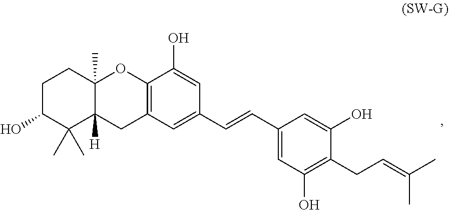 [SMILES: CC(C)=CCc1c(O)cc(/C=C/c2cc(O)c3c(c2)C[C@@H]2C(C)(C)[C@H](O)CC[C@@]2(C)O3)cc1O]
schweinfurthin K of formula (SW-K), 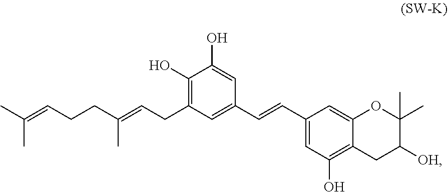 [SMILES: CC(C)=CCC/C(C)=C/Cc1cc(/C=C/c2cc(O)c3c(c2)OC(C)(C)C(O)C3)cc(O)c1O]
and schweinfurthin R of formula (SW-R) 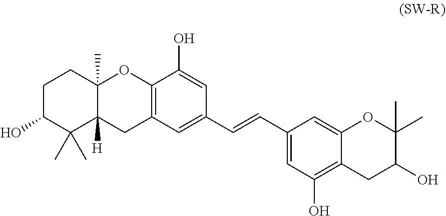 [SMILES: CC1(C)Oc2cc(/C=C/c3cc(O)c4c(c3)C[C@@H]3C(C)(C)[C@H](O)CC[C@@]3(C)O4)cc(O)c2CC1O]
characterized in that it comprises a step of using mappain of formula (IV)

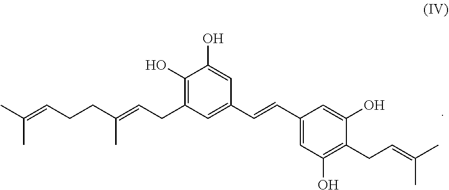 [SMILES: CC(C)=CCC/C(C)=C/Cc1cc(/C=C/c2cc(O)c(CC=C(C)C)c(O)c2)cc(O)c1O]

2. Process according to claim 1, wherein it comprises a step (i) of protecting mappain of formula (IV) by placing mappain in contact with a compound of formula PG-X in which X is a nucleofugal group chosen from halogens, tosylate, mesylate, nonaflate, phosphate, sulfamate or triflate, PG being a phenol-protecting group, to obtain a compound of formula (III)

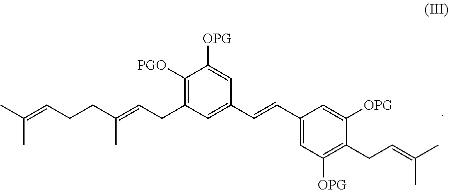

3. Process according to claim 1 wherein the protecting groups PG of the phenols are chosen from alkoxyalkyl, alkoxyalkoxyalkyl, alkoxyaryl, alkyl, and trialkylsilyl groups.

4. Process according to claim 2 wherein the placing of the mappain of formula (IV) in contact with the compound of formula PG-X is preceded by the placing of the mappain in contact with a base chosen from organic and mineral bases.

5. Process according to claim 1 wherein it comprises a step (ii) of epoxidation of a compound of formula (III) 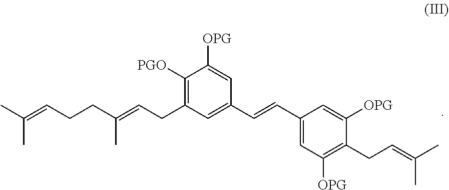
in the presence of an oxidizing agent to obtain at least one compound chosen from the group consisting of the compound of formula (II-G) 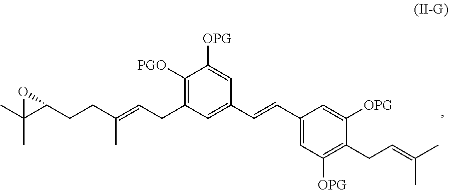
the compound of formula (II-K) 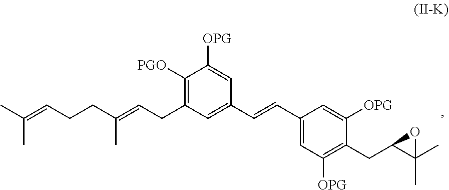
and optionally the compound of formula (II-R)

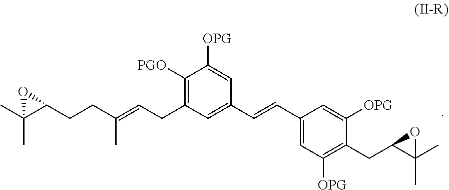

6. Process according to claim 5, wherein the epoxidation is a Shi epoxidation, performed in the presence of a Shi reagent of formula (1)

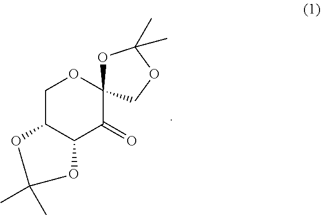

7. Process according to claim 6 wherein the oxidizing agent is present in a mole ratio relative to the compound of formula (III) ranging from 7 to 200.

8. Process according to claim 1 wherein it comprises a step (iii) of cyclization in the presence of a Lewis acid, of at least one compound chosen from the compounds of formulae (II-G) 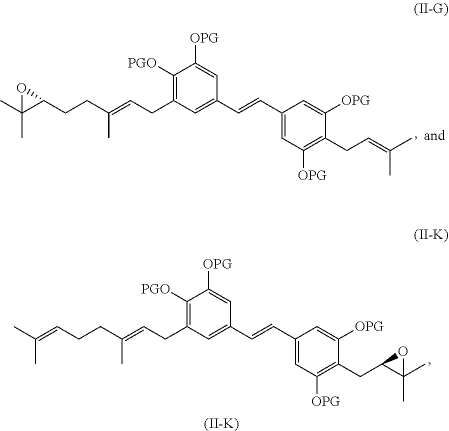
to obtain the compound of formula (I-G) 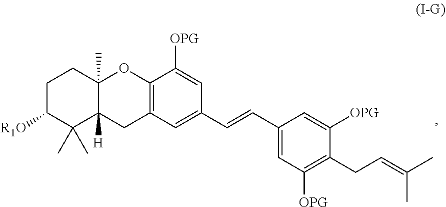
the compound of formula (I-K) 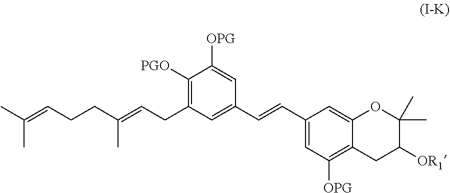
the by-product of formula (I-L) 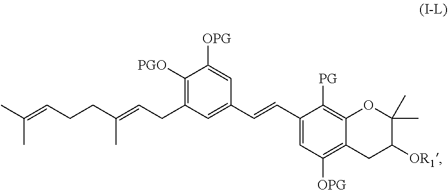
or a mixture thereof, in which R₁ and R₁′ are independently chosen from the group consisting of hydrogen and a group PG and in which PG is a phenol-protecting group.

9. Process according to claim 1 wherein it comprises a step (iii-R) of cyclization, in the presence of a Lewis acid, of a compound of formula (II-R) 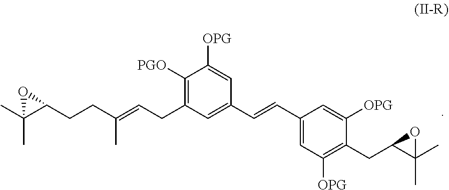
to obtain the compound of formula (I-R) 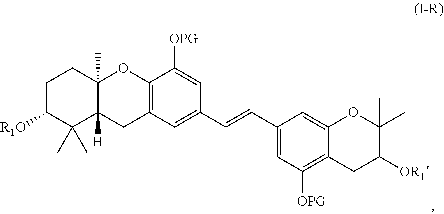
and the by-product of formula (I-S) 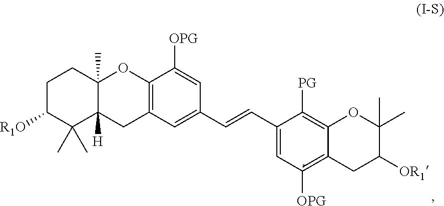
in which PG is a phenol-protecting group, and in which R₁ and R₁′ are independently chosen from the group consisting of hydrogen and PG.

10. Process according to claim 9 wherein the Lewis acid is chosen from the group consisting of boron trifluoride sources, dialkylaluminium chlorides, fluoro alcohols, and montmorillonite or metal trifluoromethanesulfonates.

11. Process for preparing schweinfurthin G of formula (SW-G) 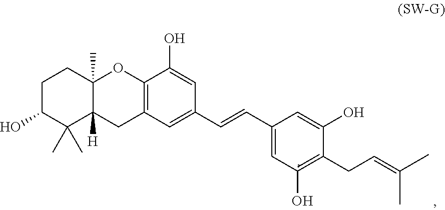 [SMILES: CC(C)=CCc1c(O)cc(/C=C/c2cc(O)c3c(c2)C[C@@H]2C(C)(C)[C@H](O)CC[C@@]2(C)O3)cc1O]
according to claim 1 wherein it comprises a step of deprotection of a compound of formula (I-G) 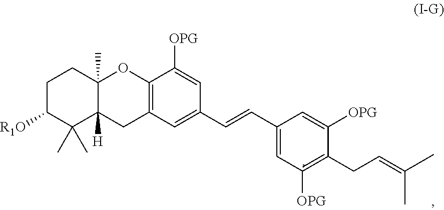
where PG is a phenol protecting group.

12. Process for preparing schweinfurthin K of formula (SW-K) 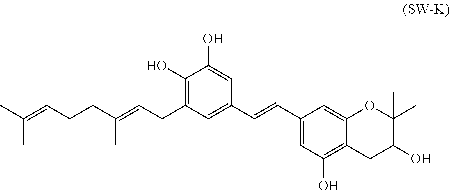 [SMILES: CC(C)=CCC/C(C)=C/Cc1cc(/C=C/c2cc(O)c3c(c2)OC(C)(C)C(O)C3)cc(O)c1O]
according to claim 1 wherein it comprises a step of deprotection of a compound of formula (I-K) 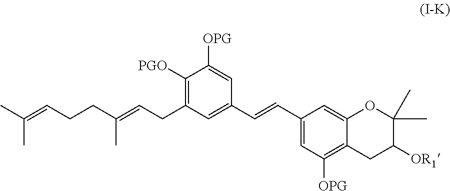
where PG is a phenol protecting group.

13. Process for preparing schweinfurthin R of formula (SW-R) 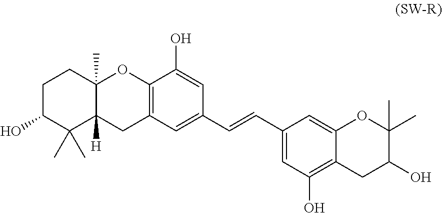 [SMILES: CC1(C)Oc2cc(/C=C/c3cc(O)c4c(c3)C[C@@H]3C(C)(C)[C@H](O)CC[C@@]3(C)O4)cc(O)c2CC1O]
according to claim 1 wherein it comprises a step of deprotection of a compound of formula (I-R) 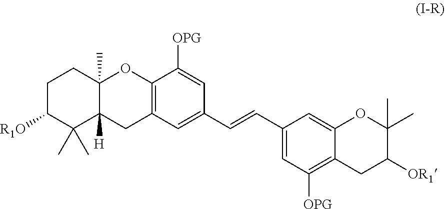
where PG is a phenol protecting group.

14. A method of using
schweinfurthin G of formula (SW-G), 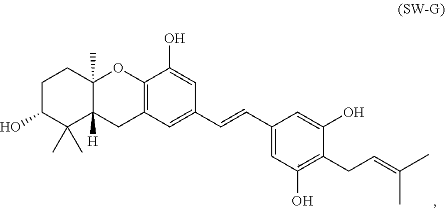 [SMILES: CC(C)=CCc1c(O)cc(/C=C/c2cc(O)c3c(c2)C[C@@H]2C(C)(C)[C@H](O)CC[C@@]2(C)O3)cc1O]
schweinfurthin K of formula (SW-K), 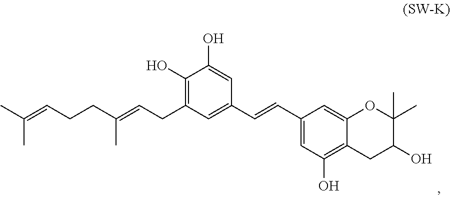 [SMILES: CC(C)=CCC/C(C)=C/Cc1cc(/C=C/c2cc(O)c3c(c2)OC(C)(C)C(O)C3)cc(O)c1O]
and schweinfurthin R of formula (SW-R) 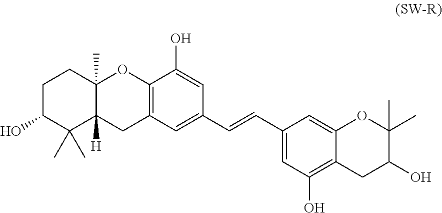 [SMILES: CC1(C)Oc2cc(/C=C/c3cc(O)c4c(c3)C[C@@H]3C(C)(C)[C@H](O)CC[C@@]3(C)O4)cc(O)c2CC1O]
obtained via a process according to claim 1, as a synthetic intermediate for obtaining a schweinfurthin derivative.

15. Process according to claim 1 further comprising preparing a pharmaceutical composition comprising a schweinfurthin chosen from the compounds of formulae (SW-G), (SW-K) and (SW-R), and pharmaceutically acceptable excipients.

16. Synthetic intermediate chosen from the compounds of formula 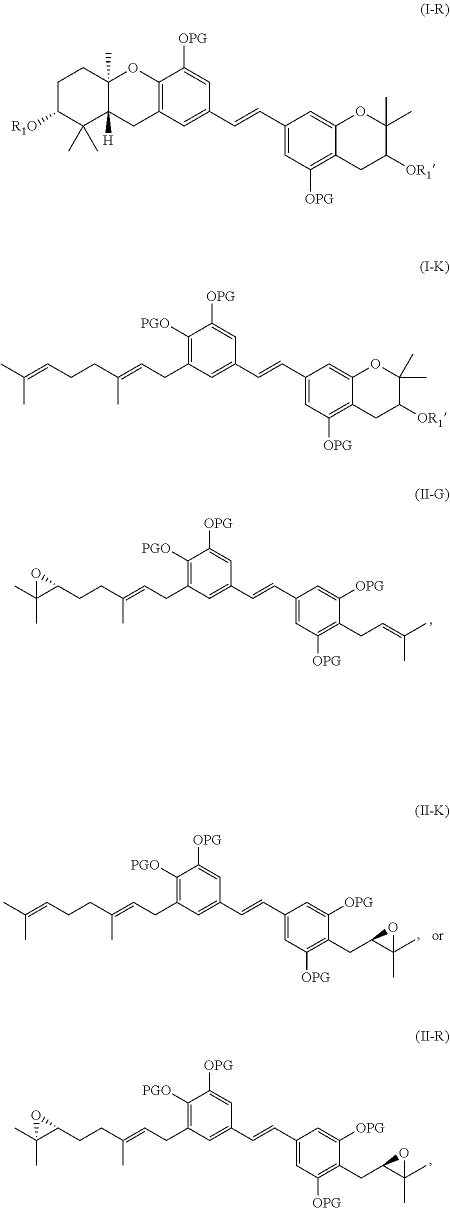
in which PG is a phenol protecting group and in which R₁ and R₁′ are independently chosen from the group consisting of hydrogen and a group PG.

17. Compound of formula (SW-R)

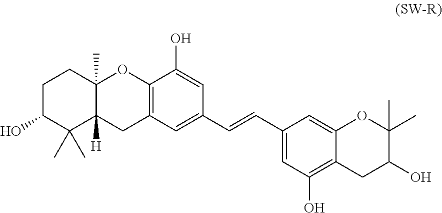 [SMILES: CC1(C)Oc2cc(/C=C/c3cc(O)c4c(c3)C[C@@H]3C(C)(C)[C@H](O)CC[C@@]3(C)O4)cc(O)c2CC1O]

18. Process according to claim 4 wherein the mineral base is chosen from potassium carbonate, sodium hydride, potassium hydroxide, sodium hydroxide and caesium carbonate, and the organic base chosen from pyridine, N,N-diisopropylethylamine and 4-dimethylaminopyridine.

19. A method of using
schweinfurthin G of formula (SW-G), 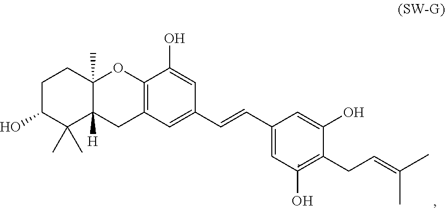 [SMILES: CC(C)=CCc1c(O)cc(/C=C/c2cc(O)c3c(c2)C[C@@H]2C(C)(C)[C@H](O)CC[C@@]2(C)O3)cc1O]
schweinfurthin K of formula (SW-K). 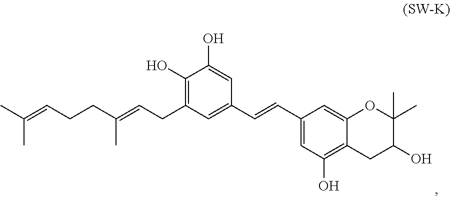 [SMILES: CC(C)=CCC/C(C)=C/Cc1cc(/C=C/c2cc(O)c3c(c2)OC(C)(C)C(O)C3)cc(O)c1O]
and schweinfurthin R of formula (SW-R) 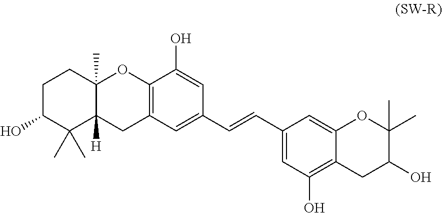 [SMILES: CC1(C)Oc2cc(/C=C/c3cc(O)c4c(c3)C[C@@H]3C(C)(C)[C@H](O)CC[C@@]3(C)O4)cc(O)c2CC1O]
obtained via a process according to claim 1 as a synthetic intermediate for obtaining vedelianin, schweinfurthin E, or schweinfurthin F.
